(19) United States
(12) Patent Application Publication
(10) Pub. No.: US 2016/0279263 A1
(43) Pub. Date: Sep. 29, 2016

(54) DRUG DELIVERY COMPOSITIONS AND METHODS TARGETING P-GLYCOPROTEIN

(71) Applicant: CORNELL UNIVERSITY, Ithaca, NY (US)

(72) Inventors: David PUTNAM, Ithaca, NY (US); Lindsey CRAWFORD, Bennington, VT (US)

(73) Assignee: CORNELL UNIVERSITY, Ithaca, NY (US)

(21) Appl. No.: 14/442,550

(22) PCT Filed: Nov. 14, 2013
(86) PCT No.: PCT/US2013/069983

§ 371 Date: May 13, 2015

Related U.S. Application Data
(60) Provisional application No. 61/726,180, filed on Nov. 14, 2012.

(51) Int. Cl. A61K 47/48; A61K 9/00

(52) U.S. Cl. CPC A61K 47/48915; A61K 9/0053; A61K 47/48907; A61K 47/48061

(57) ABSTRACT

A composition having general structure (1); wherein the P-gp substrate is a substrate for P-glycoprotein; the linker is a biocompatible polymeric moiety; the drug-loaded carrier comprises a biocompatible framework carrying at least one drug; and the straight line shown in Formula (1) between the drug-loaded carrier and linker represents a first bond, and the straight line shown in Formula (1) between the linker and P-gp substrate represents a second bond. Also described herein are pharmaceutical compositions containing the above compositions, as well as methods for using these compositions for targeted delivery of a drug to cells expressing higher levels of P-glycoprotein compared to other cells in a mammal, for the treatment of various diseases or conditions, such as cancer and neurological conditions.

### CROSS REFERENCE TO RELATED APPLICATIONS

This application claims priority from U.S. Provisional Application No. 61/726,180, filed Nov. 14, 2012, the entire contents of which are incorporated herein by reference.

### STATEMENT REGARDING FEDERALLY SPONSORED RESEARCH OR DEVELOPMENT

This invention was made with Government Support under Contract No. W81XWH-10-1-0915, awarded by the United States Department of Defense. The Government has certain rights in this invention.

### FIELD OF THE DISCLOSURE

This invention relates to compositions and methods for delivery of therapeutic substances to tissues and cells expressing P-glycoprotein (P-gp). The delivery compositions disclosed herein are generally composed of a drug-loaded carrier linked to a P-gp substrate.

### BACKGROUND ART

P-glycoprotein (P-gp) is an ATP-binding cassette membrane efflux protein that actively transports small molecules across the cell membrane. It is expressed in both healthy (e.g., blood brain barrier, and gastrointestinal endothelium) and diseased (e.g., multidrug resistant cancers) tissues (ECKFORD, et al., Chemical Reviews, 109:2989-3011 (2009); SZAKÁCS, et al., Nature Reviews, 5:219-34 (2006); BORST, et al., J. Natl. Cancer Inst., 92:1295-1302 (2000)). The X-ray structure of P-gp was solved and revealed two distinct binding pockets (each on their own respective half of the protein). One binding pocket is hydrophobic and the other charged, which provides diversity among substrates of P-gp (ALLER, et al., Science, 323: 1718-22 (2009)). Although general characteristics of P-gp substrates are known, such as high lipophilicity, the protein is fairly promiscuous and transports a surprisingly wide range of structures (ALLER, et al., Science, 323: 1718-22 (2009); KIM, Drug Metabolism Reviews, 34: 47-54 (2002)). The clinical ramifications of P-gp expression in cancers are well documented. P-gp expulses various classes of drugs, leading to resistance to a multitude of substrates including chemotherapeutics, antibiotics, dyes and protease inhibitors (ECKFORD, et al., Chemical Reviews, 109: 2989-3011 (2009); KIM, Drug Metabolism Reviews, 34: 47-54 (2002)). The literature has documented examples of drug targeting with antibodies against P-gp expressing cell types (MATSUO, et al., J. Controlled Release 77: 77-86 (2001); IWAHASHI, et al., Cancer Res., 53: 5475-5482 (1993)) and of attempts to inhibit P-gp mediated efflux of substrates (THOMAS, et al., Cancer Control, 10: 159-65 (2003)). However, to date the clinical impacts of these approaches have been yet to be fully realized. Probes for evaluating the functionality of P-gp have also been documented (PIRES, et al., Biochemistry, 45: 11695-702 (2006); PIRES, et al., Mol. Pharmacol., 7: 92-100 (2009)).

### SUMMARY OF THE DISCLOSURE

The instant disclosure is directed to P-gp-targeting compositions carrying a drug, as well as their use in treating or preventing a disease or condition. The P-gp-targeting composition includes a P-gp substrate linked to a drug-loaded carrier, wherein the P-gp substrate provides the P-gp targeting ability of the composition. The composition can be conveniently depicted by the following general structure:

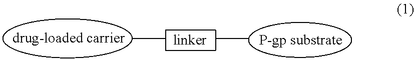

In Formula (1) above, the component labeled as “P-gp substrate” is a substrate for P-glycoprotein; the component labeled as “linker” is a biocompatible polymeric moiety; the component labeled as “drug-loaded carrier” includes a biocompatible framework carrying at least one drug; the straight line shown in Formula (1) between the drug-loaded carrier and linker represents a first bond, and the straight line shown in Formula (1) between the linker and P-gp substrate represents a second bond.

In another aspect, the instant disclosure is directed to methods for targeted delivery of a drug to cells expressing higher levels of P-glycoprotein compared to other cells in a mammal, wherein the method includes administering to the mammal a pharmaceutically effective amount of the P-gp-targeting composition described above. The cells being targeted can be, for example, cancerous cells, in which case the drug is typically an anti-cancer drug. In some embodiments, the cancerous cells are multi-drug resistant (MDR) cancerous cells. In other embodiments, the cells being targeted are epithelial cells of the blood-brain barrier, in which case the drug is typically a neuroactive drug; and in some embodiments, a neuroactive drug that has an ability to cross a blood-brain barrier. In other embodiments, the cells being targeted are cells of the gastrointestinal epithelium, in which case the drug may be a gastrointestinal drug or any drug for which systemic circulation is desired.

### BRIEF DESCRIPTION OF THE DRAWINGS

FIG. 1. Conjugation reaction of biotin-PEG-NH22 to the P-gp substrate, rhodamine 123. All reactions were carried out in dimethylformamide (DMF) in the presence of excess triethylamine. A 3:1 mol ratio of rho 123:NH2 was used. Purification was conducted by dialysis against distilled water, and final chemical structure determined by ¹H NMR.

FIG. 2. ¹H NMR showing 94% conjugation. (*) the peak from biotin (**) peak from rhodamine 123, integrated peak area used to calculate percent conjugation.

FIG. 3. Fluorescent polystyrene nanoparticle used for initial targeting studies. Targeted form includes a rho123 end group. Untargeted control contains a CH3 end group.

FIG. 4. Schematic diagram of a circular parallel-plate flow chamber. Cells are grown on a glass surface over which a gasket is placed allowing the flow of reagents over the top of the cells. Attachment of beads to the cell surface is quantified by use of a fluorescent microscope positioned above the flow chamber visible window.

FIG. 5. Western blot showing P-gp over expression in MCF7-P-gp(+) cells compared to positive control CACO-2 cells and negative control MCF7-P-gp(−) cells.

FIG. 6. FACS data in initial in vitro targeting studies. (a) MCF7-P-gp(−) cells (b) MCF7-P-gp(−) cells with control beads (c) MCF7-P-gp(−) cells with rho 123 beads; (d) MCF7-P-gp(+) cells (e) MCF7-P-gp(+) cells with control beads; (f) MCF7-P-gp(+) cells with rho 123 beads.

FIG. 7. Flow chamber data from flow chamber studies. The results show that control beads (without rhodamine) do not adhere to either P-gp expressing or P-gp expressing MCF-7 cells, whereas beads containing rhodamine123 adhere to P-gp expressing MCF-7 cells, but not to non-expressing cells. Collectively these data demonstrate the feasibility of using P-gp substrates to target MDR(+) tumors.

FIG. 8. Binding kinetics profile of rhodamine-targeted beads to MCF-7/Mdr1 cells.

FIG. 9. Scheme 1. Transition of rhodamine123 from open to close spiro form with the addition of base (adapted from ADAMCZYK, et al., J. Bioorganic & Medicinal Chemistry Letters, 10: 1539-41 (2000)). Scheme 2. Synthesis of mPEG-Rho123 by nucleophilic attack at the 2′ position.

FIG. 10. ¹H NMR (600 MHz, DMSO-d₆) of proposed structure of mPEG-Rho123 conjugate.

FIG. 11. Stejskals-Tanner plots of diffusion ordered NMR. (▪) Rho123 peaks at 6.14 and 6.16 ppm, (▴) Rho123 peaks at 6.09 and 6.10 ppm, (□) mPEG peak at 3.17 ppm, (Δ) mPEG peak at 3.44 ppm, (♦) DMSO-d₆ a) free Rho123 and unconjugated mPEG-NH₂. Free Rho123 diffuses faster than mPEG-NH₂. b) conjugated mPEG-Rho123. Covalent attachment evident from equal diffusion of Rho123 peaks and mPEG peaks.

FIG. 12. FACS analysis of mPEG-Rho 123 conjugate (a-g), free Rho 123 (i-p) and mPEG-NH₂ (h) accumulation in MDR and WT cells. Shaded spectra represent MDR cells with either mPEG-Rho 123 or Rho 123 added at the appropriate condition. Dashed spectra represent WT cells with either mPEG-Rho 123 or Rho 123 added at the appropriate condition. Solid line spectra represent MDR cells with no mPEG-Rho123 or Rho123. Dotted lines represent WT cells with no mPEG-Rho123 or Rho123. Conditions are: 0 μM verapamil (a and i), 5 μM verapamil (b and j), 25 μM verapamil (c and k), 50 μM verapamil (d and 1), 75 μM verapamil (e and m), 100 μM verapamil (f and n), 4° C. (g and o) and 15 μM mPEG-NH₂ (h and p). Efflux from MDR cells of mPEG-Rho123 conjugate follows the same pattern as free Rho123 with varying concentration of verapamil. In both cases MDR spectra approached WT spectra with increasing verapamil concentration. At 4° C. accumulation of mPEG-Rho 123 conjugate significantly decreased in WT cells compared to 0 μM verapamil condition. Addition of 15 μM mPEG-NH₂ with free Rho123 at 0 μM verapamil followed the same efflux pattern of free Rho123 alone at 0 μM verapamil.

FIG. 13. a) WT to MDR accumulation ratio based on mode fluorescence. Dark gray bars represent mPEG-Rho123 conjugate and white bars represent Rho123. Both mPEG-Rho123 conjugate and free Rho123 showed an increase in accumulation ratio with increasing verapamil concentration. There was no significant decrease in accumulation ratio for free Rho 123 in the presence of 15 μM mPEG-NH₂. b) Mode fluorescence of mPEG-Rho 123 conjugate. Black bars represent fluorescence in MDR cells and light gray bars are fluorescence in WT cells. There was a significant decrease in WT accumulation at 4° C. due to inhibition of endocytosis. c) Mode fluorescence of Rho123. Black bars represent fluorescence in MDR cells and light gray bars are fluorescence in WT cells. The increase in MDR mode fluorescence was due to inhibition of P-gp at 4° C. All bars are an average of 3 replicates. * represents statistical significance of p<0.01, # not significant compared to free Rho123 with 0 μM verapamil.

FIG. 14. Scheme 3. Synthesis of mPEG-PLA by ring opening polymerization of D,L-lactide.

FIG. 15. ¹H NMR (400 MHz, DMSO-d₆) of mPEG-PLA. Peaks a and b are from the mPEG component and peaks c and d are from PLA component.

FIG. 16. TEM images of 5000-73,129 PEG-PLA nanoparticles. Scale bar is 200 nm.

FIG. 17. Initial biodistribution data using europium nanospheres. a) nanospheres with mPEG and b) nanospheres with PEG-Rho123. Europium was able to be detected against background tissue fluorescence. Each time point represents the average of n=5. Error bars are representative of standard deviations.

### DETAILED DESCRIPTION

This disclosure is directed to a drug delivery vehicle targeting P-gp, composed of a drug-loaded carrier, which is linked through a biocompatible polymeric moiety as a linker, to a P-glycoprotein (P-gp) substrate. After the delivery vehicle is administered to a mammalian subject (i.e., generally a human), and once the drug-loaded carrier reaches the tissues expressing P-gp, the P-gp substrate in some embodiments diffuses into the cell membrane and is then recognized by P-gp and ejected from the cell, in which case the remaining portion of the drug delivery vehicle becomes tethered to the tissue site; and in other embodiments, the substrate simply binds to the P-gp and the delivery vehicle becomes localized to the tissue. In both sets of embodiments, the drug can be released locally. The delivery system of this invention is particularly useful for delivery of compounds to tissues and cells expressing high levels of P-gp, such as multi drug resistant cancerous cells, blood brain barrier, and gastrointestinal epithelium, for therapeutic treatment of cancer, CNS disorders and GI disorders, among others.

P-Gp Targeting Drug Delivery Compositions

In one aspect, the invention is directed to P-gp targeting compositions in which a drug-loaded carrier is linked, via a linker, to a P-gp substrate. The P-gp targeting composition can be conveniently depicted by the following general structure:

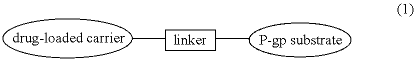

In Formula (1) above, the straight line shown in Formula (1) between the drug-loaded carrier and linker represents a first bond, and the straight line shown in Formula (1) between the linker and P-gp substrate represents a second bond. The bonds are independently of any type that can be maintained (i.e., will not degrade or be disrupted) in biological tissue at least up to the point that the P-gp substrate binds to P-gp. Thus, the first and second bonds should be of sufficient strength, such as, independently, a covalent, ionic, or affinity bond.

Each of the components provided in Formula (1) may represent a single entity or a multiplicity of such entities. For example, although Formula (1) depicts a single drug-loaded carrier, Formula (1) includes the possibility of a multiplicity (e.g., two, three, or more) drug-loaded carriers being separately attached to the linker, as long as the linker maintains a suitable length where no entity is bound in order that the drug-loaded carrier and P-gp substrate are suitably separated. In the case of multiple drug-loaded carriers, the straight line representing the first bond can represent multiple bonds. Similarly, the linker provided in Formula (1) may represent a single linker or a multiplicity of linkers, and the P-gp substrate provided in Formula (1) may represent a single P-gp substrate or a multiplicity of P-gp substrates. In the case of multiple P-gp substrates, the straight line representing the second bond can represent multiple bonds.

As the entire composition depicted in Formula (1) is designed to be administered into living subjects, the entire composition, including all of its components, should be biocompatible. The term “biocompatible”, as used herein, refers to the characteristic of certain substances to not induce a substantially adverse physiological response (e.g., acute immunological or toxicological response) in a subject being treated, wherein the term “substantially adverse physiological response” generally refers to a physiological response of such acuteness that the welfare of the subject or the efficacy of the treatment could be compromised, as could be determined by one skilled in the medical arts. Thus, a determination of biocompatibility is distinct from determining side effects, since side effects are generally an accepted aspect of any drug. As understood in the art, the term “biocompatible” is a relative term, since some degree of immune response and/or toxicity is common for most materials internalized into a subject, including those materials generally deemed biocompatible. In some embodiments, the term “biocompatible” indicates that the composition or a component thereof fails to elicit any detectable immunological or toxicological response from the subject being administered the composition.

Drug-Loaded Carrier

The drug-loaded carrier includes a biocompatible framework that carries at least one drug. The biocompatible framework can have any structure suitable for carrying a drug. The drug can be carried (i.e., incorporated) in an inner portion, on an outer surface, or a combination thereof.

In one embodiment, the biocompatible framework possesses a substantially hollow interior portion surrounded by a biocompatible material, which may or may not be porous. In the foregoing embodiment, the drug can reside in the hollow interior portion, in which case the surrounding biocompatible material encapsulates the drug. If the encapsulating material is porous, the pores may be nanoporous, mesoporous, or macroporous, or a combination thereof, as long as the pores are small enough to maintain a substantial portion (e.g., at least 50, 60, 70, 80, 90, or 95%) or all of the encapsulated drug within the encapsulating material at least during the time the composition is being carried in the subject and until it binds to a P-gp target. Once docked at the P-gp site, the pores may serve to slowly release the drug.

In another embodiment, the biocompatible framework possesses a structure in which the drug can intercalate, i.e., the drug can be embedded in, absorbed to, or conjugated to the biocompatible framework. The framework in which the drug can intercalate may or may not include a hollow interior portion. Thus, in some embodiments, the drug may be encapsulated while also being intercalated in another portion of the biocompatible framework. Such intercalating structures for drug delivery are well known in the art.

In particular embodiments, the biocompatible framework is constructed of a biocompatible polymer, which may have an organic or inorganic backbone. The encapsulating polymer can be, for example, a polyhydroxyacid biopolyester, polysaccharide, polyacrylate, polymethacrylate, polyalkyleneglycol, polyphosphazene, polyanhydride, polyacetal, poly(ortho esters), polyurea, polyurethane, polyamide, poly(amino acid), polyphosphoester, or a co-polymer thereof. Other polymer chemistries are possible, such as polycarbonates, polypyrroles, polyoxazoline, and polysiloxanes. A comprehensive review of these and other biocompatible polymers and their use in drug delivery is provided in G. Vilar, et al., “Polymers and Drug Delivery Systems”, Current Drug Delivery, vol. 9, no. 4, 2012, pp. 1-28, which is herein incorporated by reference in its entirety.

The term “polyhydroxyacid biopolyester” (i.e., “biopolyester”) is meant to encompass all of those biocompatible polymers, as known in the art, that possess ester bonds, many of which are microbially produced or are known to be biodegradable. Two particular subclasses of biopolyesters considered herein are the poly(α-hydroxy acid)s and poly(hydroxyalkanoates). The poly(hydroxyalkanoates) generally refer to polyesters of non-α-hydroxy acids, such as polyesters of β-, γ-, δ-, and ε-hydroxy acids.

Some examples of α-hydroxy acids that can be polymerized into a biopolyester include lactic acid, glycolic acid, 2-hydroxybutyric acid, malic acid, tartaric acid, and mandelic acid. Any biocompatible and suitably hydrophilic homopolymer or copolymer containing a polymerized block of any one, two, or more of these α-hydroxy acids are considered herein. Some examples of poly(α-hydroxy acid)s include polylactic acid (PLA), polyglycolic acid (PGA), poly(2-hydroxybutyric acid), poly(malic acid), poly(tartaric acid), and poly(mandelic acid), as well as co-polymers thereof, such as a PLA-PGA (PLGA) copolymer. A copolymer containing one or more poly(α-hydroxy acid) blocks may also include one or more non-α-hydroxy acid polymer blocks, such as one or more polyalkyleneglycol blocks (e.g., PLA-PEO, PGA-PEO, and PLGA-PEO copolymers). Such polymers and copolymers are well known in the art, as evidenced by, for example, F. Rancan, et al., “Investigation of Polylactic Acid (PLA) Nanoparticles as Drug Delivery Systems for Local Dermatotherapy”, Pharmaceutical Research, vol. 26, no. 8, August 2009, pp. 2027-2036; Riley, et al., “Colloidal stability and drug incorporation aspects of micellar-like PLA-PEG nanoparticles.” Colloids and Surfaces B: Biointerfaces, vol. 16, 1999, pp. 147-159; Y. Yamamoto, et al., “Long-circulating poly(ethylene glycol)-poly(D,L-lactide) block copolymer micelles with modulated surface charge”, J. Control Release, 2001 Nov. 9; 77(1-2):27-38; R. Z. Xiao, et al., “Recent advances in PEG-PLA block copolymer nanoparticles”, International Journal of Nanomedicine, vol. 2010:5, November 2010, pp. 1057-1065; H. Xia, et al., “Penetratin-functionalized PEG-PLA nanoparticles for brain drug delivery”, Int. J. Pharm., 436(1-2); October 2012, pp. 840-850; S. Abdollahi, et al., “PLGA- and PLA-Based Polymeric Nanoparticles for Antimicrobial Drug Delivery”, Biomedicine International, vol. 3, no. 1, 2012; I. Bala, et al., “PLGA nanoparticles in drug delivery: the state of the art”, 21(5), 2004, pp. 387-422; and Y. Wang, et al., “Chitosan-modified PLGA nanoparticles with versatile surface for improved drug delivery”, AAPS PharSciTech, 14(2), June 2013, pp. 585-592; all of which are herein incorporated by reference in their entirety.

Some examples of hydroxyalkanoic acids that can be polymerized into a poly(hydroxyalkanoate) biopolyester include 3-hydroxypropionate (HP), 3-hydroxybutyrate, 4-hydroxybutyrate, 3-hydroxyvalerate, 4-hydroxyvalerate, 5-hydroxyvalerate, ε-caprolactone, 3-hydroxyhexanoate, and 3-hydroxyoctanoate. Any biocompatible and suitably hydrophilic homopolymer or copolymer containing a polymerized block of any one, two, or more of these hydroxyalkanoic acids are considered herein. Some examples of poly(hydroxyalkanoates) include poly(3-hydroxypropionate), poly(3-hydroxybutyrate), poly(4-hydroxybutyrate), poly(3-hydroxyvalerate), poly(4-hydroxyvalerate), poly(5-hydroxyvalerate), poly(s-caprolactone), poly(3-hydroxyhexanoate), and poly(3-hydroxyoctanoate), as well as co-polymers thereof, such as a copolymer of 3-hydroxybutyrate and 3-hydroxyvalerate (i.e., “PHB-co-PHV”), or copolymer of 3-hydroxypropionate and 3-hydroxybutyrate (i.e., “PHP-co-PHB”), or copolymer of 3-hydroxypropionate and 3-hydroxyvalerate (i.e., “PHP-co-PHV”), or copolymer of 3-hydroxybutyrate and ε-caprolactone (i.e., “PHB-co-PCL”), or copolymer of 3-hydroxypropionate and ε-caprolactone (i.e., “PHP-co-PCL”). A copolymer containing one or more poly(hydroxyalkanoate) blocks may also include one or more non-hydroxyalkanoate polymer blocks, such as one or more polyalkyleneglycol blocks (e.g., PHP-PEO, PHB-PEO, PHV-PEO, and PCL-PEO copolymers) or one or more poly(α-hydroxy acid) blocks (e.g., PLA-PHP, PLA-PHB, PLA-PHV, PLA-PCL, PGA-PHP, PGA-PHB, PGA-PHV, PGA-PCL, PLGA-PHP, PLGA-PHB, PLGA-PHV, and PLGA-PCL copolymers). Such polymers and copolymers are well known in the art, as evidenced by, for example, U.S. Pat. No. 8,563,281; U.S. Pat. No. 8,551,512; U.S. Pat. No. 6,534,599; U.S. Application Pub. No. 2013/0065046; C. S. K. Reddy, et al., “Polyhydroxyalkanoates: an overview”, Bioresource Technology, vol. 87, no. 2, April 2003, pp. 137-146; G. A. Nobes, et al., “Polyhydroxyalkanoates: materials for delivery systems”, Drug Delivery, 5(3), 1998, pp. 167-177; Y.-C. Xiong, et al., “Application of Polyhydroxyalkanoates Nanoparticles as Intracellular Sustained Drug-Release Vectors, Journal of Biomaterials Science, 21, (2010) pp. 127-140; S. Kabilan, et al., “Pseudomonas sp. as a Source of Medium Chain Length Polyhydroxyalkanoates for Controlled Drug Delivery: Perspective”, International Journal of Microbiology, vol. 2012 (2012), article ID 317828, 10 pages; K. Sudesh, et al., “Synthesis, structure and properties of polyhydroxyalkanoates: biological polyesters”, Progress in Polymer Science, vol. 25, no. 10, December 2000, pp. 1503-1555; C. W. Pouton, et al., “Biosynthetic polyhydroxyalkanoates and their potential in drug delivery”, Advanced Drug Delivery Reviews, vol. 18, no. 2, January 1996, pp. 133-162; M. I. Ré, et al., “New PHB/PHPE microspheres obtained from Burkholderia cepacia as biodegradable drug delivery systems for photodynamic therapy”, Minerva Biotecnologica, March 2006, 18(1), pp. 3-9; C. Errico, et al., “Poly(hydroxyalkanoates)-Based Polymeric Nanoparticles for Drug Delivery”, Journal of Biomedicine and Biotechnology, Volume 2009 (2009), Article ID 571702, 10 pages; all of which are herein incorporated by reference in their entirety.

The biocompatible framework may also be or include a polysaccharide. The polysaccharide may be based on, for example, dextran, dextran sulfate, hyaluronic acid, alginate, heparin, chondroitin sulfate, pectin, pullulan, amylose, a cyclodextrin, a chitosan (e.g., chitosan, carboxymethyl chitosan, glycol chitosan, N-trimethyl chitosan, N-triethyl chitosan), cellulose, carboxymethyl cellulose, or glucomannan, or a combination or co-polymer thereof. Such polymers and their use in drug delivery are well known in the art, as evidenced by, for example, Z. Liu, et al., “Polysaccharides-based nanoparticles as drug delivery systems”, Advanced Drug Delivery Reviews, vol. 60, no. 15, 2008, pp. 1650-1662; and Saravanakumar G. et al., “Polysaccharide-based nanoparticles: a versatile platform for drug delivery and biomedical imaging”, Curr. Med. Chem., 19(19), 2012, pp. 3212-3229, all of which are herein incorporated by reference in their entirety.

The biocompatible framework may also be or include a vinyl addition polymer. Some examples of such polymers include polyacrylic acid, polyacrylate salt, polymethacrylic acid, polymethacrylate salt, poly(methyl acrylate), poly(methyl acrylate), poly(ethyl acrylate), poly(methyl methacrylate), poly(2-hydroxyethyl acrylate), poly(2-hydroxyethyl methacrylate), polyvinyl alcohol, polyvinyl acetate, and polyacrylamides, including N-substituted versions thereof, such as poly(N-isopropylacrylamide), as well as combinations and co-polymers thereof. Such polymers and their use in drug delivery are well known in the art, as evidenced by, for example, Garay-Jimenez, J. C., et al., “A convenient method to prepare emulsified polyacrylate nanoparticles from powders for drug delivery applications”, Bioorg. Med. Chem. Lett., 21(15), 2011, pp. 4589-4591; S. Benita, et al., “Polyacrylate resin (Eudragit Retard) microcapsules as a controlled release drug delivery system improved non-solvent addition phase separation process”, Journal of Microencapsulation, vol. 2, no. 3, 1985, pp. 207-222; R. Kumar, et al., “Biodegradable polymethacrylic acid grafted psyllium for controlled drug delivery systems”, Frontiers of Chemical Science and Engineering, vol. 7, no. 1, 2013, pp. 116-122; Y. Zhang, et al., “Biocompatible and degradable poly(2-hydroxyethyl methacrylate) based polymers for biomedical applications”, Polym. Chem., 3, 2012, pp. 2752-2759; Hsuie, G. H., et al., “Poly(2-hydroxyethyl methacrylate) film as a drug delivery system for pilocarpine”, Biomaterials, 22(13), 2001, pp. 1763-1769; and Sutar, P. B., et al., “Development of pH sensitive polyacrylamide grafted pectin hydrogel for controlled drug delivery system”, J. Mater. Sci. Mater. Med., 19(6), 2008, pp. 2247-2253, all of which are herein incorporated by reference in their entirety.

The biocompatible framework may also be or include a polyalkylene glycol. The polyalkylene glycol can be any of the polyalkylene glycols known in the art, such as polyethylene glycol, polypropylene glycol, or poly(trimethylene glycol), or a combination or co-polymer thereof. Such polymers and their use in drug delivery are well known in the art, as evidenced by, for example, K. Knop, et al., “Poly(ethylene glycol) in Drug Delivery: Pros and Cons as Well as Potential Alternatives”, Angewandte Chemie, vol. 49, no. 36, 2010, pp. 6288-6308; A. Mero, et al., “A Biodegradable Polymeric Carrier Based on PEG for Drug Delivery”, Journal of Bioactive and Compatible Polymers, May 2009, vol. 24 no. 3 220-234; R. B. Greenwald, “Poly(ethylene glycol) Anticancer Drug Delivery Systems”, PRHS, vol. 21, no. 2, 2002, pp. 113-121; S. Parveen, et al., “Long circulating chitosan/PEG blended PLGA nanoparticle for tumor drug delivery”, Eur. J. Pharmacol., 670(2-3), 2011, pp. 372-383; E. Locatelli, et al., “Biodegradable PLGA-b-PEG polymeric nanoparticles: synthesis, properties, and nanomedical applications as drug delivery system”, Journal of Nanoparticle Research, 14:1316, November 2012; H. Ocal, et al., “Novel pentablock copolymer (PLA-PCL-PEG-PCL-PLA) based nanoparticles for controlled drug delivery: Effect of copolymer compositions on the crystallinity of copolymers and in vitro drug release profile from nanoparticles”, Colloid and Polymer Science, 291:5, 2013, pp. 1235-1245; and T. Ishihara, et al., “Polymeric nanoparticles encapsulating betamethasone phosphate with different release profiles and stealthiness”, International Journal of Pharmaceutics, 375:1-2, 2009, pp. 148-54, all of which are herein incorporated by reference in their entirety.

The biocompatible framework may also be or include a polyphosphazene. As known in the art, polyphosphazenes are polymers that possess an inorganic backbone constructed of alternating phosphorus and nitrogen atoms, with variable side chains attached to the phosphorus atoms. The polyphosphazenes have the particular advantage of synthetic flexibility, ease of fabrication, and matrix permeability. Such polymers and their use in drug delivery are well known in the art, as evidenced by, for example, S. Lakshmi, et al., “Biodegradable polyphosphazenes for drug delivery applications”, Advanced Drug Delivery Reviews, vol. 55, no. 4, 2003, pp. 467-482; I. Teasdale, et al., “Polyphosphazenes: Multifunctional, Biodegradable Vehicles for Drug and Gene Delivery”, Polymers, 5, 2013, pp. 161-187; and Y. Lemmouchi, et al., “Biodegradable polyphosphazenes for drug delivery”, Macromolecular Symposia, 123, 1997, pp. 103-112, all of which are herein incorporated by reference in their entirety.

The biocompatible framework may also be or include a polyanhydride polymer, which may be saturated or unsaturated, and either aliphatic or aromatic. Such polymers and their use in drug delivery are well known in the art, as evidenced by, for example, C. T. Laurencin, et al., “Bioerodible polyanhydrides for antibiotic drug delivery: in vivo osteomyelitis treatment in a rat model system”, J. Orthop. Res., 11(2), 1993, pp. 256-262; J. P. Jain, et al., “Polyanhydrides as localized drug delivery carrier: an update”, Expert Opin. Drug Deliv., 5(8), 2008, pp. 889-907; and H. B. Rosen, et al., “Bioerodible Polyanhyrides for Controlled Drug Delivery”, Biomaterials, vol. 4, April 1983, pp. 131-133; all of which are herein incorporated by reference in their entirety.

The biocompatible framework may also be or include a polyacetal. Polyacetal polymers and their use in drug delivery are well known in the art, as evidenced by, for example, J.-K. Kim et al., “Novel pH-sensitive polyacetal-based block copolymers for controlled drug delivery”, Int. J. Pharm., 401(1-2), 2010, pp. 79-86; S. E. Paramonov, et al., “Fully acid-degradable biocompatible polyacetal microparticles for drug delivery”, Bioconjug. Chem., 19(4), April 2008, pp. 911-919; and M. J. Vincent, et al., “Polyacetal-diethylstilboesterol: A Polymeric Drug Designed for pH-triggered Activation”, Journal of Drug Targeting, vol. 12, no. 8, September 2004, pp. 491-501; all of which are herein incorporated by reference in their entirety.

The biocompatible framework may also be or include a poly(ortho ester). Poly(ortho ester) polymers and their use in drug delivery are well known in the art, as evidenced by, for example, U.S. Pat. No. 6,524,606 (“Bioerodible Polyorthoesters Containing Amine Groups”); J. Heller, et al., “Poly(ortho esters): synthesis, characterization, properties and uses”, Adv. Drug. Deliv. Rev., 54(7), 2002, pp. 1015-1039; J. Heller, et al., “Poly(ortho esters)—from concept to reality”, Biomacromolecules, 5(5), 2004, pp. 1625-1632; S. Einmahl, et al., “A New Poly(Ortho Ester)-Based Drug Delivery System as an Adjunct Treatment in Filtering Surgery”, Investigative Ophthalmology & Visual Science, March 2001, Vol. 42, No. 3, pp. 695-700; and C. Shih, et al., “Drug delivery from catalysed erodible polymeric matrices of poly(ortho ester)s”, Biomaterials, 5(4), July 1984, pp. 237-240; all of which are herein incorporated by reference in their entirety.

The biocompatible framework may also be or include a polyurea. Polyurea polymers and their use in drug delivery are well known in the art, as evidenced by, for example, U.S. Pat. No. 8,529,880 (“Biodegradable polyurethane/urea compositions”); W. He, et al., “Surfactant-Free One-Step Synthesis of Dual-Functional Polyurea Microcapsules: Contact Infection Control and Drug Delivery”, Advanced Functional Materials, vol. 22, no. 19, 2012, pp. 4023-4031; F. Xiang, et al., “One-Pot Synthesis for Biocompatible Amphiphilic Hyperbranched Polyurea Micelles”, Macromolecules, 4611), 2013, pp. 4418-4425; G. Morral-Ruiz, et al., “Design of biocompatible surface-modified polyurethane and polyurea nanoparticles”, Polymer, 53, 2012, pp. 6072-6080; and P. Cass, et al., “Synthesis and evaluation of degradable polyurea block copolymers as siRNA delivery agents”, Acta. Biomater., 9(9), 2013, pp. 8299-8307; all of which are herein incorporated by reference in their entirety.

The biocompatible framework may also be or include a polyamide. Polyamide polymers and their use in drug delivery are well known in the art, as evidenced by, for example, U.S. Pat. No. 8,277,841 (“Polyamide rate-modulated monolithic drug delivery system”); I. Gachard, et al., “Drug delivery from nonpeptidic α-amino acid containing polyamides”, Polymer Bulletin, vol. 38, no. 4, April 1997, pp. 427-431; and D. Crespy, et al., “Preparation of Nylon 6 Nanoparticles and Nanocapsules by Two Novel Miniemulsion/Solvent Displacement Hybrid Techniques”, Macromolecular Chemistry and Physics, vol. 208, no. 5, March 2007, pp. 457-466; all of which are herein incorporated by reference in their entirety.

The biocompatible framework may also be or include a polyurethane. Polyurethane polymers and their use in drug delivery are well known in the art, as evidenced by, for example, U.S. Pat. No. 8,529,880 (“Biodegradable polyurethane/urea compositions”); J. Y. Cherng, et al., “Polyurethane-based drug delivery systems”, Int. J. Pharm., vol. 450, no. 1-2, June 2013, pp. 145-162; L. Zhou, et al., “Synthesis and Characterization of pH-Sensitive Biodegradable Polyurethane for Potential Drug Delivery Applications”, Macromolecules, 44(4), 2011, pp. 857-864; M. Mandru, et al., “Characteristics of polyurethane-based sustained release membranes for drug delivery”, Central European Journal of Chemistry, April 2013, vol. 11, no. 4, pp 542-553; F. Borcan, et al., “Synthesis and preliminary in vivo evaluations of polyurethane microstructures for transdermal drug delivery”, Chemistry Central Journal, 6:87, August 2012; S.-G. Kang, et al., “Paclitaxel-polyurethane film for anti-cancer drug delivery: Film characterization and preliminary in vivo study”, Macromolecular Research, vol. 18, no. 7, July 2010, pp. 680-685; and R. S. Harisha, et al., “Controlled release of 5-flurouracil from biomedical polyurethanes”, J. Chem. Sci., vol. 122, no. 2, March 2010, pp. 209-216; all of which are herein incorporated by reference in their entirety.

The biocompatible framework may also be or include a poly(amino acid), i.e., polypeptide. The poly(amino acid) can be derived from any known natural or unnatural amino acid. Some examples of poly(amino acids) include poly-γ-glutamic acid, polyaspartic acid, polyserine, polythreonine, polylysine, polyglutamine, polyasparagine, polyarginine, and polycysteine, as well as copolymers thereof. Poly(amino acid) polymers and their use in drug delivery are well known in the art, as evidenced by, for example, A. Lalatsa, “Amphiphilic poly(L-amino acids)—new materials for drug delivery”, J. Control Release, 161(2), 2012, pp. 523-36; S. R. Yoon, et al., “Charge-conversional poly(amino acid)s derivatives as a drug delivery carrier in response to the tumor environment”, J. Biomed. Mater. Res. A, 100(8), August 2012, pp. 2027-2033; B. Tian, et al., “Polypeptide-based vesicles: formation, properties and application for drug delivery”, J. Mater. Chem., 22, 2012, pp. 17404-17414; J. Ding, et al., “Facile preparation of a cationic poly(amino acid) vesicle for potential drug and gene co-delivery”, Nanotechnology, vol. 22, no. 49, 2011; K. Osada, et al., “Polymeric micelles from poly(ethylene glycol)-poly(amino acid) block copolymer for drug and gene delivery”, Journal of the Royal Society Interface, vol. 6, no. suppl. 3, Jun. 6, 2009, S325-S339; and B. Manocha, et al., “Production and characterization of gamma-polyglutamic acid nanoparticles for controlled anticancer drug release”, Crit. Rev. Biotechnol., 28(2), 2008, pp. 83-99; all of which are herein incorporated by reference in their entirety.

The biocompatible framework may also be or include a polyphosphoester. Polyphosphoester polymers and their use in drug delivery are well known in the art, as evidenced by, for example, Z. Zhao, et al., “Polyphosphoesters in drug and gene delivery”, Adv. Drug Deliv. Rev., 55(4), 2003, pp. 483-499; and J. Zhou, et al., “Poly(ethylene oxide)-block-Polyphosphoester-graft-Paclitaxel Conjugates with Acid-Labile Linkages as a pH-Sensitive and Functional Nanoscopic Platform for Paclitaxel Delivery”, Adv. Healthc. Mater., 2013 Aug. 30, doi: 10.1002/adhm.201300235 (online); all of which are herein incorporated by reference in their entirety.

In another embodiment, the biocompatible framework is a liposome. As well known in the art, a liposome has a lipid bilayer structure formed by the ordered assembly of amphiphilic molecules. In an aqueous environment, the liposome possesses a hydrophobic layer having inner and outer surfaces that are hydrophilic. Thus, if the drug is suitably hydrophilic, the drug may be encapsulated in an interior portion of the liposome or be attached to an outer surface thereof, whereas, if the drug is suitably hydrophobic, the drug may be intercalated within the hydrophobic layer of the liposome. The liposome can have any of the compositions well known in the art, such as a phosphatidylcholine phospholipid composition, phosphatidylethanolamine phospholipid composition, phosphatidylinositol phospholipid composition, or phosphatidylserine phospholipid composition. Liposome compositions and their use in drug delivery are well known in the art, as evidenced by, for example, U.S. Pat. No. 8,304,565; U.S. Pat. No. 8,329,213; U.S. Application Pub. No. 2008/113015; Medina O. P., et al., “Targeted liposomal drug delivery in cancer”, Curr. Pharm. Des., 2004; 10(24):2981-9; Allen T. M., et al., “Liposomal drug delivery systems: from concept to clinical applications”, Adv. Drug Deliv. Rev., 2013 January; 65(1):36-48; and W. Gao, et al., “Liposome-like nanostructures for drug delivery”, J. Mater. Chem. B, 2013; all of which are herein incorporated by reference in their entirety.

In another embodiment, the biocompatible framework is a micelle. As well known in the art, a micelle is distinct from a liposome in that it is not a bilayer structure and possesses a hydrophobic interior formed by the ordered interaction of amphiphilic molecules. Thus, a drug of sufficient hydrophobicity may be intercalated or encapsulated within the micellular structure, while a drug of sufficient hydrophilicity may be attached to the outer surface of the micelle. The micelle can be constructed of any of the numerous biocompatible compositions known in the art, such as a PEG-PLA or PEG-PCL composition. Micellular compositions and their use in drug delivery are well known in the art, as evidenced by, for example, U.S. Pat. No. 8,529,917; U.S. Pat. No. 8,367,113; T. Riley, et al., “Colloidal stability and drug incorporation aspects of micellar-like PLA-PEG nanoparticles.” Colloids and Surfaces B: Biointerfaces, vol. 16, 1999, pp. 147-159; Croy and Kwon, “Polymeric micelles for drug delivery”, Curr. Pharm. Design, 12:4669-4684 (2006); M.-C. Jones, et al., “Polymeric micelles—a new generation of colloidal drug carriers”, Eur. J. Pharmaceutics Biopharmaceutics, 48:101-111 (1999); Y. Yamamoto, et al., “Long-circulating poly(ethylene glycol)-poly(D,L-lactide) block copolymer micelles with modulated surface charge”, J. Control Release, 2001 Nov. 9; 77(1-2):27-38; and X. Yang et al., “Folate-functionalized polymeric micelles for tumor targeted delivery of a potent multidrug-resistance modulator FG020326”, Journal of Biomedical Materials Research Part A, 2008, 86(1), pp. 48-60; all of which are herein incorporated by reference in their entirety.

In another embodiment, the biocompatible framework is a dendrimer. The dendrimer can be any of the dendrimers known in the art that can suitably carry a drug in a biological system, such as the well-known poly(amidoamine) (PAMAM) dendrimers, amino acid-based dendrimers, ester-containing (biodegradable) dendrimers, and glycodendrimers. Dendrimer compositions and their use in drug delivery are well known in the art, as evidenced by, for example, M. Ina, et al., “Dendrimer: a novel drug delivery system”, Journal of Drug Delivery & Therapeutics, 2011, 1(2): pp. 70-74; S. Bai, et al., “Recent progress in dendrimer-based nanocarriers”, Crit. Rev. Ther. Drug Carrier Syst., 23(6), 2006, pp. 437-495; E. R. Gillies, et al., “Dendrimers and dendritic polymers in drug delivery”, Drug Discovery Today, 10(1), 2005, pp. 35-43; and S. H. Medina, et al., “Dendrimers as Carriers for Delivery of Chemotherapeutic Agents”, Chem. Rev., 109, 2009, pp. 3141-3157, all of which are herein incorporated by reference in their entirety.

In another embodiment, the biocompatible framework is a metal organic framework. The metal organic framework can be any of the metal organic frameworks known in the art that can suitably carry a drug in a biological system. Metal organic framework compositions and their use in drug delivery are well known in the art, as evidenced by, for example, U.S. Pat. No. 8,569,407 (“Biodegradable material composed of a polymer comprising a porous metal-organic framework”); P. Horcajada, et al., “Porous metal-organic-framework nanoscale carriers as a potential platform for drug delivery and imaging”, Nature Materials, 9, 2010, pp. 172-178; R. C. Huxford, et al., “Metal-Organic Frameworks as Potential Drug Carriers”, Curr. Opin. Chem. Biol., 14(2), April 2010, pp. 262-268; H.-N. Wang, et al., “Stepwise assembly of metal-organic framework based on a metal-organic polyhedron precursor for drug delivery”, Chem. Commun., 47, 2011, pp. 7128-7130; and S. Keskin, et al., “Biomedical Applications of Metal Organic Frameworks”, Industrial & Engineering Chemistry Research, 50(4), 2011, pp. 1799-1812; all of which are herein incorporated by reference in their entirety.

In some embodiments, the biocompatible framework is a solid (i.e., non-hollow) bead or particle on which the drug is conjugated on an outer surface thereof. The bead can be constructed of any of the compositions, well known in the art, acceptable for administration into a living subject. The bead can be composed of, for example, any of the compositions described above, or alternatively, polystyrene, polyethylene, polypropylene, or a metallic composition, which may be magnetic. Such compositions and their use in drug delivery are well known in the art, as evidenced by, for example, G. Tiwari, et al., “Drug delivery systems: an updated review”, Int. J. Pharm. Investig., 2(1), January-March 2012, pp. 2-11; H. Kaur, et al., “Transportation of drug-(polystyrene bead) conjugate by actomyosin motor system”, J. Biomed. Nanotechnol., 6(3), 2010, pp. 279-286; and A. Nussinovitch, “Beads as Drug Carriers”, Polymer Macro- and Micro-Gel Beads: Fundamentals and Applications, 2010, pp. 191-230; all of which are herein incorporated by reference in their entirety.

In particular embodiments, the biocompatible framework is in the form of a nanoparticle or microparticle, which may be hollow or non-hollow (solid). In some embodiments, the particles have a substantially spherical shape, while in other embodiments, the particles are substantially non-spherical, e.g., oval, globular, or a film. As used herein, the term “nanoparticle” generally refers to a particle having a size of at least 1 nm and less than 1 micron, e.g., a particle having a size of precisely, about, at least, greater than, up to, or less than, for example, 1, 2, 3, 4, 5, 10, 15, 20, 25, 30, 35, 40, 45, 50, 60, 70, 80, 90, 100, 110, 120, 130, 140, 150, 160, 170, 180, 190, 200, 250, 300, 350, 400, 450, 500, 600, 700, 800, or 900 nm, or a size within a range bounded by any two of the foregoing values, wherein the term “about” is generally no more than ±10, 5, 4, 3, 2, or 1% of a number. As used herein, the term “microparticle” generally refers to particles having a size of at least 1 micron (1 μm), e.g., a particle having a size of precisely, about, at least, greater than, up to, or less than, for example, 2, 3, 4, 5, 10, 15, 20, 25, 30, 35, 40, 45, 50, 60, 70, 80, 90, 100, 110, 120, 130, 140, 150, 160, 170, 180, 190, or 200 μm, or a size within a range bounded by any two of the foregoing values. For particles having a substantially spherical shape, any of the sizes provided above can be regarded as a diameter of the particle. For particles having a substantially non-spherical shape, any of the sizes provided above can be regarded as an average size (i.e., average of the particle's three dimensions) or a longest dimension. The sizes, compositions, and particular applications of various nanoparticles and microparticles used in drug delivery are well known in the art, such as described in: D. S. Kohane, “Microparticles and nanoparticles for drug delivery”, Biotechnol. Bioeng., 2007, 96(2), pp. 203-209; M. N. V. R. Kumar, “Nano and Microparticles as Controlled Drug Delivery Devices”, J. Pharm. Pharmaceut. Sci., 3(2), 2000, pp. 234-258; J. Siepmann, “Microparticles used as drug delivery systems”, Progress in Colloid and Polymer Science, vol. 133, 2006, pp. 15-21; all of which are herein incorporated by reference in their entirety. In specific embodiments, nanoparticles having an average diameter of about 50-200 nm are used in this invention.

The synthesis of particles of any of the compositions described above is well known in the art, and may include, for example, nanoprecipitation, emulsion-diffusion, double emulsification, emulsion-coacervation, polymer-coating, and layer-by-layer methods. These and other methods are well known in the art, as evidenced by the numerous references provided above, as well as, for example, U.S. Pat. No. 8,546,521 (“Method for fabricating nanoparticles”); U.S. Pat. No. 8,207,290 (“Methods and systems for generating nanoparticles”); U.S. Pat. No. 8,420,123 (“Drug loaded polymeric nanoparticles and methods of making and using same”); U.S. Pat. No. 8,367,113 (“Polymers for functional particles”); U.S. Pat. No. 8,318,208 (“Drug loaded polymeric nanoparticles and methods of making and using same”); C. E. Mora-Huertas, et al., “Polymer-based nanocapsules for drug delivery”, International Journal of Pharmaceutics, vol. 385, no. 1-2, January 2010, pp. 113-142; Soppimath, et al., “Biodegradable polymeric nanoparticles as drug delivery devices”, Journal of Controlled Release, 2001, 1-20, vol. 70; Vauthier, et al., “Methods for the preparation and manufacture of polymeric nanoparticles”, Pharmaceutical Research, (2008), 34 pages; Wei et al., “Biodegradable poly(ε-caprolactone)-poly(ethylene glycol) copolymers as drug delivery system”, International Journal of Pharmaceutics, (2009) vol. 381, no. 1, pp. 1-18; Xie et al., “Fabrication of PLGA nanoparticles with a fluidic nanoprecipitation system”, Journal of Nanobiotechnology, (2010) vol. 8, 7 pages; and H. N. Kim, et al., “Enzymatic synthesis of a drug delivery system based on polyhydroxyalkanoate-protein block copolymers”, Chem. Comm. (Camb), 46, 2009, pp. 7104-7106; all of which are herein incorporated by reference in their entirety.

Linker

In Formula (1), the linker can be composed of any of the biocompatible compositions known in the art, and which maintains linkage between the biocompatible framework and P-gp substrate during and at least up to binding to cellular P-gp in a living subject. For purposes of the invention, the linker should be of suitable length and suitably free of steric hindrance from the drug-loaded biocompatible framework to at least partially, or in some cases, completely, traverse a cell membrane, which is typically at least 3, 4, 5, 6, 7, 8, 9, or 10 nm thick. To achieve this, the linker should be at least 3 nm in length, which corresponds to approximately 4, 5, or 6 ethylene oxy units. In different embodiments, the linker is at least 3 nm in length, and up to or less than, for example, 4, 5, 6, 7, 8, 9, 10, 12, 15, 20, 30, 40, or 50 nm in length. Moreover, to reduce steric hindrance and the ability of the linker to penetrate a cell membrane, the linker is typically a single linear (i.e., unbranched) polymeric chain. In some embodiments, to ensure adequate solubility in biological tissue and permeation of the cell membrane, the linker also possesses hydrophilic groups. The hydrophilic groups can be any of those groups, well known in the art, that confer an enhanced degree of aqueous solubility. Some common hydrophilic groups include ether (i.e., carbon-oxygen-carbon), hydroxy (—OH), carboxylic acid (—COOH), ester (—C(O)O—) carboxylate salt (—COO⁻M⁺, wherein M⁺ represents a suitable countercation), and ammonium groups.

In some embodiments, the linker is composed, at least partly or completely, of a polyalkylene oxide segment, which may have any of the polyalkylene oxide compositions described above for the biocompatible framework. In particular embodiments, the linker is composed, at least partly or completely, of a segment of polyethylene oxide (PEG), polypropylene oxide (PPO), or a copolymer thereof. In different embodiments, the polyalkylene oxide linker includes at least 4, 5, or 6, and up to or less than 7, 8, 9, 10, 12, 15, 20, 25, 30, 40, 50, 60, 70, 80, 90, 100, 110, 120, 130, 140, 150, 160, 170, 180, 190, 200, 250, or 300 alkylene oxide units. In embodiments where the linker is a copolymer of a polyalkylene oxide and non-polyalkylene oxide (e.g., poly(α-hydroxy acid) or polyhydroxyalkanoate, such as PLA, PGA, PHP, PHB, or PHV), then the foregoing number of polyalkylene oxide units can be taken as total polymeric units, with any suitable number of non-polyalkylene oxide units deducting from said number of polyalkylene oxide units.

A linker can be attached to the biocompatible framework and to the P-gp substrate by means well known in the art, such as by grafting or covalent binding by use of a reactive functional group. For example, in the particular case of polyalkylene oxides (and more particularly, PEG), a wide variety of derivatized forms are either commercially available and can be synthesized by means well known in the art. Moreover, numerous bifunctional crosslinking forms of linear and branched segments of such polymers are known. In one embodiment, an activated ester (e.g., N-hydroxysuccinimide, or NHS) form of PEG is reacted with either the biocompatible framework or the P-gp substrate, wherein the biocompatible framework and/or P-gp substrate possess functional groups (e.g., amino or hydroxy) that are reactive with the activated ester on the PEG derivative. In another embodiment, a biotinylated form of PEG is reacted with an avidin- or streptavidin-functionalized form of the biocompatible framework or P-gp substrate. In another embodiment, a positively-charged ammonium-functionalized PEG derivative is reacted with a biocompatible framework and/or P-gp substrate having negatively-charged groups thereon. In yet another embodiment, an amino-derivatized PEG derivative is reacted with a biocompatible framework and/or P-gp substrate having amino-reactive groups thereon, such as carboxylic acid, ester, activated ester, alkyl halide, or aldehyde groups. Various activating molecules may also be used to facilitate the conjugation. For example, carbonyldiimidazole (CDI) or a carbodiimide (e.g., N,N′-dicyclohexylcarbodiimide, or DCC) may be used to enhance the coupling between a carboxylic acid group and an amino group to form an amide bond between the two groups.

The preparation and conjugation of such linkers are well known in the art, as evidenced by, for example, U.S. Pat. No. 7,030,278 (“Polyethylene(glycol) derivatives with proximal reactive groups”); U.S. Application Pub. No. 2009/0285780 (“PEG linker compounds and biologically active conjugates thereof”); PCT International Publication WO2006/116742 (“Fluorescent nanoparticles conjugated to antibodies via a PEG linker”); M. K. Yu, et al., “Targeting Strategies for Multifunctional Nanoparticles in Cancer Imaging and Therapy”, Theranostics, 2(1), 2012, pp. 3-44; M. De, et al., “Applications of Nanoparticles in Biology”, Adv. Mater., 20, 2008, pp. 4225-4241; W. K. Lee, et al., “Preparation and characterization of biodegradable nanoparticles entrapping immunodominant peptide conjugated with PEG for oral tolerance induction”, J. Control Release, 2005, 105(1-2), pp. 77-88; Y. Liu, et al., “A strategy for precision engineering of nanoparticles of biodegradable copolymers for quantitative control of targeted drug delivery”, Biomaterials, 31(35), 2010, pp. 9145-9155; S. Dziadek, et al., “A novel linker methodology for the synthesis of tailored conjugate vaccines composed of complex carbohydrate antigens and specific TH-cell peptide epitopes”, Chemistry, 14(19), 2008, pp. 5908-5917; “Effect of Polyethylene Glycol Linker Chain Length of Folate-Linked Microemulsions Loading Aclacinomycin A on Targeting Ability and Antitumor Effect In vitro and In vivo”, Clin. Cancer Res., Mar. 1, 2005, 11, pp. 2018-2025; A. Gabizon, et al., “Targeting Folate Receptor with Folate Linked to Extremities of Poly(ethylene glycol)-Grafted Liposomes: In Vitro Studies”, Bioconjugate Chem., 10(2), 1999, pp. 289-298; and C. Schmidtke, et al., “Amphiphilic, cross-linkable diblock copolymers for multifunctionalized nanoparticles as biological probes”, Nanoscale, 5(16), 2013, pp. 7433-7444; all of which are herein incorporated by reference in their entirety.

P-gp Substrate

In Formula (1), the term “P-gp substrate” refers to any moiety that binds P-glycoprotein (P-gp). In some embodiments, a P-gp substrate enters the cell and binds to P-gp from the interior of the cell. In other embodiments, a P-gp substrate binds P-gp from the exterior of the cell. In some embodiments, the P-gp substrate can be an inhibitor of P-gp, i.e., a compound that interferes with P-gp efflux activity. For purposes of the invention, the P-gp substrate should retain its ability as a substrate for P-gp when conjugated to the linker-(biocompatible framework) moiety. In some embodiments, the P-gp substrate possesses an affinity for P-gp that is better than micromolar (e.g., less than or equal to 1 micromolar). In other embodiments, the P-gp substrate possesses an affinity for P-gp that is evidenced in a dissociation constant (Kᵢ) of up to or less than, for example, 1 mM, 500 nM, 400 nM, 300 nM, 200 nM, 100 nM, 90 nM, 80 nM, 70 nM, 60 nM, or 50 nM, or an affinity within a range bounded by any two of the foregoing values. To place the Kᵢ value in context, the Kᵢ value for LY335979, a highly potent modulator of P-gp, is known to be 59 nM, i.e., A. H. Dantzig, et al., “Reversal of multidrug resistance by the P-glycoprotein modulator, LY335979, from the bench to the clinic”, Curr. Med. Chem., 8(1), January 2001, pp. 39-50, the contents of which are herein incorporated in their entirety. The P-gp substrate preferably exhibits these properties along with a lack of significant inhibition other members of the MRP family (e.g., MRP1) and BCRP.

Using the known and continually improved criteria for determining whether a substance is a P-gp substrate or non-substrate, a large number of P-gp substrates have been and continue to be identified, i.e., Lee, J. S., et al., (1994) “Rhodamine efflux patterns predict P-glycoprotein substrates in the National Cancer Institute drug screen”, Mol. Pharmacol., 46, 627-38; Gombar, V. K., et al., (2004) “Predicting P-glycoprotein substrates by a quantitative structure-activity relationship model”, J. Pharm. Sci., 93, 957-68; Z. Bikadi, et al., “Predicting P-Glycoprotein-Mediated Drug Transport Based On Support Vector Machine and Three-Dimensional Crystal Structure of P-glycoprotein”, PLoS One, 6(10), 2011, e25815; S. Seeland, et al., “On-line identification of P-glycoprotein substrates by monitoring of extracellular acidification and respiration rates in living cells”, Biochim. Biophys. Acta., 1808(7), July 2011, pp. 1827-1831; A. A. El Ela, et al., “Identification of P-glycoprotein substrates and inhibitors among psychoactive compounds—implications for pharmacokinetics of selected substrates”, J. Pharm. Pharmacol., 56(8), August 2004, pp. 967-975; C. Chang, et al., “Rapid Identification of P-glycoprotein Substrates and Inhibitors”, Drug Metab. Dispos., 34(12), December 2006, pp. 1976-1984; and H. Thomas, et al., “Overcoming multidrug resistance in cancer: an update on the clinical strategy of inhibiting P-glycoprotein”, Cancer Control, vol. 10, no. 2, March/April 2003, pp. 159-165; all of which are herein incorporated by reference in their entirety. For purposes of the invention, the P-gp substrate should also exhibit at least some selectivity for P-gp relative to other cellular components. Preferably, the P-gp substrate selectively targets cells having a higher density of P-gp compared to other cells in a living subject.

In some embodiments, the P-go substrate is a small molecule compound. By “small molecule” it is meant small organic hydrocarbon compounds having a molecular weight of less than 15 kD, 12 kD, 10 kD, or even 8 kD.

In particular embodiments, the P-gp substrates include the rhodamine class of fluorophores, such as Rhodamine 6G and Rhodamine 123. The use of rhodamine compounds as P-gp substrates is well known in the art, as evidenced by, for example, Kajikawa, T., et al., (1999) “Role of P-glycoprotein in distribution of rhodamine 123 into aqueous humor in rabbits”, Current Eye Research, 18, pp. 240-6; Zastre, J., et al., (2002) “Enhanced cellular accumulation of a P-glycoprotein substrate, rhodamine-123, by Caco-2 cells using low molecular weight methoxypolyethylene glycol-block-polycaprolactone diblock copolymers”, European Journal of Pharmaceutics and Biopharmaceutics, 54, pp. 299-309; Eytan, G. D., et al., (1997) “Efficiency of P-glycoprotein-mediated exclusion of rhodamine dyes from multidrug-resistant cells is determined by their passive transmembrane movement rate”, European Journal of Biochemistry, 248, pp. 104-112; Loetchutinat, C., et al., (2003) “New insights into the P-glycoprotein-mediated effluxes of rhodamines”, European Journal of Biochemistry, 270, pp. 476-485; and Nare, B., et al., (1994) “Characterization of rhodamine 123 binding to P-glycoprotein in human multidrug-resistant cells”, Mol. Pharmacol., 45, pp. 1145-52; all of which are herein incorporated by reference in their entirety. An additional advantage provided by the rhodamine class of P-gp substrates is that they can be used in fluorescence imaging during and after reaching the target.

Numerous other classes and specific types of P-gp substrates may also be suitable. Some of these are listed in U.S. Pat. No. 8,512,707, which is herein incorporated by reference in its entirety. The P-gp substrate can be, for example, darunavir, maraviroc, digoxin, loperamide, quinidine, vinca alkaloids (e.g., vinblastine or vincristine), acrivastine, talinolol, ketoconazole, zosuquidar (LY335979), nelfinavir, ritonavir, saquinavir, tacrolimus, valspodar, verapamil, elacridar, reserpine, amiodarone, azithromycin, captopril, carvedilol, clarithromycin, conivaptan, cyclosporine, diltiazem, dronedarone, dexamethasone, betamethasone, erythromycin, felodipine, itraconazole, lopinavir, quercetin, ranolazine, aliskiren, ambrisentan, colchicine, dabigatran etexilate, everolimus, fexofenadine, imitanib, lapatinib, nilotinib, posaconazole, saxagliptin, sirolimus, sitagliptin, tolvaptan, topotecan, indinavir, an anthracycline, doxorubicin, duanorubicin, epirubicin, mitxantrone, etoposide, amprenavir, ranitidine, propanalol, prazosin, methotrexate, cefazolin, cefoperazone, cerivastatin, cetirizine, or a taxane (e.g., paclitaxel or docetaxel). In some embodiments, one or more of any of the above P-gp substrates can be excluded. In other embodiments, the P-gp substrate is not an antibody or antibody fragment.

Reference is also made to U.S. Application Pub. No. 2009/0093493, which discloses existing and new compounds that function as P-gp substrates, and which is herein incorporated by reference in its entirety. The foregoing application describes 1-phenylalkoxy-2-beta-phenylethyl derivatives useful as P-gp inhibitors, all of which are herein considered as P-gp substrates.

Drug

In Formula (1), the drug can be any biological or chemical substance useful in the treatment, prevention, or diagnosis of a disease or condition. As used herein, the term “drug” is also meant to encompass a “prodrug”, i.e., a substance that is altered or activated in the living subject to become an active drug. As used herein, the term “treatment” is meant to encompass curing, amelioration, remission, or management (e.g., prevention of progression) of the symptoms or etiology of a disease or condition. The drug can be, for example, an anti-cancer (i.e., cytotoxic or anti-neoplastic or chemotherapeutic) drug, neuroactive (psychoactive) drug, gastrointestinal agent, antimicrobial (e.g., antibiotic, antiviral, antifungal, or anti-parasitic), analgesic (pain reliever), anti-inflammatory, statin, anti-diabetic drug, anti-hypertensive drug, cardiovascular drug, or anti-obesity drug.

The anticancer drug can be any drug that treats, prevents, or diagnoses cancer, as provided in, for example, S. Nussbaumer, et al., “Analysis of anticancer drugs: a review”, Talanta, 85(5), 2011, pp. 2265-2289, the contents of which are herein incorporated in their entirety. Some classes of anticancer drugs include the alkylating agents (e.g., nitrogen mustards, tetrazines, nitroureas, cisplatins, and aziridines), anti-metabolites, anti-microtubule agents, cytotoxic antibiotics, and topoisomerase inhibitors. Some examples of specific anticancer drugs include cisplatin and its derivatives (e.g., cisplatin, carboplatin, and oxaliplatin), mechlorethamine, cyclophosphamide, melphalan, busulfan, ifosfamide, chlorambucil, N-nitroso-N-methylurea (MNU), fotemustine, streptozotocin, lomustine, semustine, dacarbazine, temozolomide, mitozolomide, pemetrexed, methotrexate, the fluoropyrimidines (e.g., fluorouracil and capecitabine), deoxynucleoside analogs (e.g., fludarabine, nelarabine, cladribine, cytarabine, gemcitabine, decitabine, azacitidine, clofarabine, and pentostatin), thiopurines (e.g., thioguanine and mercaptopurine), vinca alkaloids (e.g., vincristine, vinblastine, vindesine, vinorelbine, and vinflunine), taxanes (e.g., paclitaxel and docetaxel), etoposide, teniposide, topotecan, irinotecan, doxorubicin, mitoxantrone, and the anthracyclines (e.g., bleomycin, actinomycin, plicamycin, mitomycin, doxorubicin, daunorubicin, epirubicin, and idarubicin), and combinations thereof.

The anticancer drug may alternatively be selected from: 1) alkaloids, including microtubule inhibitors (e.g., vincristine, vinblastine, and vindesine, etc.), microtubule stabilizers (e.g., paclitaxel (TAXOL®), and docetaxel, etc.), and chromatin function inhibitors, including topoisomerase inhibitors, such as epipodophyllotoxins (e.g., etoposide (VP-16), and teniposide (VM-26), etc.), and agents that target topoisomerase I (e.g., camptothecin and isirinotecan (CPT-11), etc.); 2) covalent DNA-binding agents (alkylating agents), including nitrogen mustards (e.g., mechlorethamine, chlorambucil, cyclophosphamide, ifosphamide, and busulfan (MYLERAN®), etc.), nitrosoureas (e.g., carmustine, lomustine, and semustine, etc.), and other alkylating agents (e.g., dacarbazine, hydroxymethylmelamine, thiotepa, and mitomycin, etc.); 3) noncovalent DNA-binding agents (antitumor antibiotics), including nucleic acid inhibitors (e.g., dactinomycin (actinomycin D), etc.), anthracyclines (e.g., daunorubicin (daunomycin, and cerubidine), doxorubicin (adriamycin), and idarubicin (idamycin), etc.), anthracenediones (e.g., anthracycline analogues, such as mitoxantrone, etc.), bleomycins (BLENOXANE®), etc., and plicamycin (mithramycin), etc.; 4) antimetabolites, including antifolates (e.g., methotrexate, FOLEX®, and MEXATE®, etc.), purine antimetabolites (e.g., 6-mercaptopurine (6-MP, PURINETHOL®), 6-thioguanine (6-TG), azathioprine, acyclovir, ganciclovir, chlorodeoxyadenosine, 2-chlorodeoxyadenosine (CdA), and 2′-deoxycoformycin (pentostatin), etc.), pyrimidine antagonists (e.g., fluoropyrimidines (e.g., 5-fluorouracil (ADRUCIL®), 5-fluorodeoxyuridine (FdUrd) (floxuridine)) etc.), and cytosine arabinosides (e.g., CYTOSAR® (ara-C) and fludarabine, etc.); 5) enzymes, including L-asparaginase, and hydroxyurea, etc.; 6) hormones, including glucocorticoids, antiestrogens (e.g., tamoxifen, etc.), nonsteroidal antiandrogens (e.g., flutamide, etc.), nonsteroidal antiestrogens (e.g. tamoxifen), and aromatase inhibitors (e.g., anastrozole (ARIMIDEX®), etc.); 7) platinum compounds (e.g., cisplatin and carboplatin, etc.); 8) monoclonal antibodies conjugated with anticancer drugs, toxins, and/or radionuclides, (e.g. Erbitux®, Rituxin®, Avastin® etc.); 9) biological response modifiers (e.g., interferons (e.g., IFN-α, etc.) and interleukins (e.g., IL-2, etc.), etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., batimastat, etc.); 17) angiogenesis inhibitors; 18) proteosome inhibitors (e.g., VELCADE®); 19) inhibitors of acetylation and/or methylation (e.g., HDAC inhibitors); 20) modulators of NF kappa B; 21) inhibitors of cell cycle regulation (e.g., CDK inhibitors); 22) modulators of p53 protein function; 23) inhibitors of protein kinases (e.g. Gleevec), and 23) radiation.

The neuroactive drug can be any drug that treats, prevents, or diagnoses a neurological disease or condition, as provided in, for example, E. Kumlien, et al., “Seizure risk associated with neuroactive drugs: Data from the WHO adverse drug reactions database”, Seizure: European Journal of Epilepsy, vol. 19, no. 2, March 2010, pp. 69-73, the contents of which are herein incorporated in their entirety. The neuroactive drug may be, for example, an anxiolytic, sedative, anti-convulsant, anti-depressant, serotonin-norepinephrine reuptake inhibitor (SSRI), anti-seizure, anti-addiction, hypnotic, anti-insomnia, anti-psychotic, or muscle relaxant drug, or a drug to treat a neurological condition, such as Alzheimer's, Parkinson's, or Huntington's disease. Some examples of neuroactive drugs include the benzodiazepenes (e.g., diazepam, bromazepam, clonazepam, nitrazepam, alprazolam, midazolam, clobazam, lorazepam, chlordiazepoxide, flurazepam, clorazepate, halazepam, prazepam, lormetazepam, temazepam, oxazepam, flunitrazepam, nitrazepam, nimetazepam, estazolam, adinazolam, triazolam, etizolam, tofisopam, and climazolam), the azapirones, barbiturates, propanolol, oxprenolol, maprotilene, escitaloprame, buproprione, clozapine, chlorprothiexene, amoxapine, donepezil, rivastigmine, quetiapine, trimipramine, zolpidem, chlorpromazine, flupenthixol, haloperidol, trifluoperazine, perphenazine, thiothixene, thioridazine, risperidone, zotepine, aripiprazole, olanzapine, clozapine, quetiapine, paliperidone, ziprasidone, paroxetine, fluoxetine, escitalopram, citalopram, phenelzine, duloxetine, venlafaxine, mirtazapine, sertraline, bupropion, isocarboxazid, carbamazepine, oxcarbazepine, topiramate, olanzapine, methylphenidate, dextroamphetamine, dexmethylphenidate, methamphetamine, modafinil, zopiclone, acetylcholinesterase inhibitors (e.g., tacrine, galantamine, rivastigmine, and donepezil), NMDA receptor antagonists (e.g., memantine), L-DOPA, dopamine agonists (e.g., bromocriptine, pergolide, piribedil, cabergoline, apomorphine, pramipexole, ropinirole, and lisuride), MAO-B inhibitors (selegiline and rasagline), amantadine, remacemide, and nefazodone, and combinations thereof.

The gastrointestinal agent can be any drug that treats, prevents, or diagnoses a gastrointestinal or digestive disease or condition, as provided in, for example, J. R. Parfitt, et al., “Pathological effects of drugs on the gastrointestinal tract: a review”, Hum. Pathol., 38(4), April 2007, pp. 527-536, the contents of which are herein incorporated in their entirety. Some examples of gastrointestinal agents include simethicone, atropine, metoclopramide, proton-pump inhibitors (e.g., omeprazole, rabeprazole, lansoprazole, esomeprazole, dexlansoprazole, pantoprazole, and ilaprazole), H₂ receptor blockers (e.g., ranitidine), sucralfate, baclofen, prostaglandin analogs (e.g., misoprostol), and hyoscyamine, and combinations thereof.

The antimicrobial drug can be any drug that treats, prevents, or diagnoses an infection caused by a microbe, as provided in, for example, K. Lewis, “Platforms for antibiotic discovery”, Nature Reviews Drug Discovery, vol. 12, 2013, pp. 371-387; S. Crunkhorn, “Antibacterial drugs: new antibiotics on the horizon?”, Nature Reviews Drug Discovery, 12, 2013, p. 99; E. D. Clercq, “Three decades of antiviral drugs”, Nature Reviews Drug Discovery, 6, 2007, p. 941; M. A. Thompson, et al., “Antiretroviral Treatment of Adult HIV Infection: 2012 Recommendations of the International Antiviral Society—USA Panel”, JAMA, 308(4), 2012, pp. 387-402; the contents of which are herein incorporated in their entirety. The antibiotic drug can be, for example, a penicillin, cephalosporin, polymyxin, rifamycin, lipiarmycin, quinolone, sulfonamide, tetracycline, macrolide, lincosamide, cyclic lipopeptide, oxazolidinone, or glycylcycline, and combinations thereof. The antiviral drug can be, for example, acyclovir, famciclovir, valacyclovir, oseltamivir, zanamivir, abacavir, zidovudine, rimantadine, amantadine, lamivudine, nevirapine, efavirenz, emtricitabine, zalcitabine, tenofovir, rilpivirine, azidothymidine, or stavudine, and combinations thereof. The antifungal drug can be, for example, a polyene (e.g., filipin, nystatin, amphotericin B, natamycin) or an N-heterocycle (e.g., an imidazole, triazole, or thiazole antifungal drug). The antiparasitic drug can be, for example, thiabendazole, mebendazole, pyrantel pamoate, ivermectin, albendazole, niclosamide, praziquantel, rifampin, tinidazole, and metronidazole.

The analgesic (pain reliever) drug can be any drug that treats (i.e., relieves) or prevents pain, as provided in, for example, R. B. Silverman, et al., “Analgesic R&D”, Nature Reviews Drug Discovery, 7, 711 (August 2008); and S. A. Schug, et al., “Neuraxial drug administration: a review of treatment options for anaesthesia and analgesia”, CNS Drugs, 20(11), 2006, pp. 917-933; the contents of which are herein incorporated in their entirety. Some examples of analgesic drugs include paracetamol, acetaminophen, non-steroidal anti-inflammatory drugs (i.e., NSAIDs, such as aspirin, ibuprofen, naproxen, fenoprofen, and ketoprofen), ropivacaine, levobupivacaine, bupivacaine, opioids, pregabalin, morphine, fentanyl, sufentanil, clonidine, dexmedetomidine, epinephrine, baclofen, neostigmine, ketamine, midazolam and adenosine, mefenamic acid, tolfenamic acid, propofol, lorazepam, Cox-2 inhibitors (e.g., celecoxib, parecoxib, and etoricoxib), and the conotoxin ziconotide, and combinations thereof.

The anti-inflammatory drug can be any drug that treats or prevents inflammation, as provided in, for example, K. Peterson, et al., “Drug Class Review: Nonsteroidal Antiinflammatory Drugs (NSAIDs)”, Final Update 4 Report, Oregon Health & Science University, © November 2010; and S. L. Curry, et al., “Nonsteroidal antiinflammatory drugs: a review”, J. Am. Anim. Hosp. Assoc., 41(5), September-October 2005, pp. 298-309; the contents of which are herein incorporated in their entirety. Some examples of anti-inflammatory drugs include aspirin, ketoprofen, meloxicam, etodolac, carprofen, deracoxib, and tepoxalin.

The statin drug can be any drug that treats or prevents hypercholesterolemia, or that functions to lower cholesterol in the body, as provided in, for example, Z. Reiner, “Statins in the primary prevention of cardiovascular disease”, Nature Reviews Cardiology, 10, August 2013, pp. 453-464, the contents of which are herein incorporated in their entirety. Some examples of statins include pravastatin, simvastatin, atorvastatin, fluvastatin, lovastatin, pitavastatin, and rosuvastatin.

The anti-diabetic drug can be any drug that treats or prevents diabetes (type 1 or 2), pre-diabetes, or hypoglycemia, or that functions to normalize or maintain blood sugar level or insulin level or function, or that functions to treat or prevent a diabetic complication, such as renal failure, cardiovascular disease, retinopathy, neuropathy, and ketoacidosis. Reference is made to, for example, D. Jonas, et al., “Drug Class Review: Newer Diabetes Medications, TZDs, and Combinations”, Oregon Health & Science University, © February 2011, the contents of which are herein incorporated in their entirety. Some examples of diabetic medications include insulin, meglitinides (e.g., repaglinide or nateglinide), sulfonylureas (e.g., glipizide, glimepiride, or glyburide), dipeptidy peptidase 4 (DPP-4) inhibitor (e.g., saxagliptin, sitagliptin, or linagliptin), biguanides (e.g., metformin), thiazolidinediones (e.g., rosiglitazone or pioglitazone), alpha-glucosidase inhibitors (e.g., acarbose, miglitol), amylin mimetics (e.g., pramlintide), and incretin mimetics (e.g., exenatide or liraglutide).

The anti-hypertensive drug can be any drug that treats or prevents hypertension, as provided in, for example, J. A. Cutler, et al., “Controlled clinical trials of drug treatment for hypertension. A review”, Hypertension, 13(5 Suppl), May 1989, pp. 136-144, the contents of which are herein incorporated in their entirety. Some examples of anti-hypertensive drugs include the diazide diuretics (e.g., epitizide, bendroflumethiazide, chlorothiazide, and hydrochlorothiazide), loop diuretics (furosemide, torsemide, bumetanide, and ethacrynic acid), thiazide-like diuretics (e.g., chlorthalidone, metolazone, and indapamide), the potassium-sparing diuretics (e.g., triamterene, amiloride, and spironolactone), beta blockers, alpha blockers, calcium channel blockers, ACE inhibitors (e.g., ramipril, fosinopril, captopril, enalapril, lisinopril, trandolapril, benazepril, and quinapril), renin inhibitors, angiotensin II receptor antagonists (e.g., losartan, candesartan, olmesartan, eprosartan, irbesartan, valsartan, and telmisartan), vasodilators, aldosterone antagonists (e.g., spironolactone or eplerenone), endothelin receptor blockers, and alpha-2 agonists.

The anti-obesity drug can be any drug that treats or prevents obesity, or that prevents weight gain, or that treats a complication arising from obesity, such as diabetes, pre-diabetes, or cardiovascular disease. Reference is made to, for example, G. Derosa, et al., “Anti-obesity drugs: a review about their effects and their safety”, Expert Opin. Drug Saf., 11(3), May 2012, pp. 459-471; J. G. Kang, et al., “Anti-obesity drugs: a review about their effects and their safety”, Diabetes Metab. J., 36(1), February 2012, p. 13-25; and D. Cooke, et al., “The obesity pipeline: current strategies in the development of anti-obesity drugs”, Nature Reviews Drug Discovery, 5, November 2006, pp. 919-931; the contents of which are herein incorporated in their entirety. Some examples of anti-obesity drugs include the glucagon-like peptide-1 (GLP-1) receptor agonists (e.g., exenatide and liraglutide), tesofensine, phentermine, topiramate, bupropion, naltrexone, bupropion, and zonisamide.

The cardiovascular drug can be any drug that treats or prevents a cardiovascular (e.g., heart or arterial) disease or condition, as provided in, for example, J. Wang, et al., “Unintended Effects of Cardiovascular Drugs on the Pathogenesis of Alzheimer's Disease”, PLoS One, 8(6), June 2013, the contents of which are herein incorporated in their entirety. Some examples of cardiovascular drugs include alpha-blockers (e.g., phentolamine, tolazoline, prazosin, terazosin, or idazoxan), beta-blockers (e.g., propanolol, pronethalol, dichloroisoprenaline, alprenolol, sotalol, bucindolol, nadolol, carteolol, labetalol, carvedilol, penbutolol, oxprenolol, timolol, metoprolol, acebutolol, betaxolol, atenolol, nebivolol, bisoprolol, landiolol, esmolol, celiprolol, ICI-118,551, butaxamine, or SR 59230A), calcium channel blockers (e.g., amlodipine, aranidipine, azelnidipine, barnidipine, benidipine, cilnidipine, clevidipine, diltiazem, isradipine, efonidipine, felodipine, lacidipine, lercanidipine, manidipine, nicardipine, nifedipine, nilvadipine, nimodipine, nisoldipine, nitrendipine, or pranidipine), hydralazine, furosemide, trandolopril, vasopressin receptor antagonist (e.g., conivaptan), an angiotensin, phospholipase inhibitor, TRP channel blocker, or an anti-thrombotic, i.e., anticoagulant, antiplatelet, or thrombolytic, such as adenosine diphosphate (ADP receptor inhibitors (e.g., prasugrel, ticagrelor, clopidogrel, or ticlopidine), phosphodiesterase inhibitors (e.g., cilostazol), adenosine reuptake inhibitors (e.g., dipyridamole), glycoprotein IIB/IIA inhibitors (e.g., eptifibatide, abciximab, or tirofiban), thromboxane inhibitors, coumarins (e.g., warfarin, acenocoumarol, or phenprocoumon), atromentin, phenindione, tissue plasminogen activator, anistreplase, urokinase, or streptokinase.

Pharmaceutical Compositions

In another aspect, the invention is directed to a pharmaceutical composition containing any one or more of the above-described compositions according to Formula (1) in a pharmaceutically acceptable carrier or excipient. The phrase “pharmaceutically acceptable carrier” or “pharmaceutically acceptable excipient”, as used herein, refers to a pharmaceutically acceptable material, composition or vehicle, such as a liquid (diluent or excipient) or solid filler. In the pharmaceutical composition, the compound is generally dispersed in the physiologically acceptable carrier, by either being mixed (e.g., in solid form with a solid carrier) or dissolved or emulsified in a liquid carrier. The carrier should be compatible with the other ingredients of the formulation and physiologically safe to the subject. Any of the carriers known in the art can be suitable herein depending on the mode of administration. Some examples of suitable carriers include gelatin, fatty acids (e.g., stearic acid) and salts thereof, talc, vegetable fats or oils, gums and glycols, starches, dextrans, and the like.

The pharmaceutical composition can also include one or more auxiliary agents, such as stabilizers, surfactants, salts, buffering agents, additives, or a combination thereof. The stabilizer can be, for example, an oligosaccharide (e.g., sucrose, trehalose, lactose, or a dextran), a sugar alcohol (e.g., mannitol), or a combination thereof. The surfactant can be any suitable surfactant including, for example, those containing polyalkylene oxide units (e.g., Tween 20, Tween 80, Pluronic F-68), which are typically included in amounts of from about 0.001% (w/v) to about 10% (w/v). The salt or buffering agent can be any suitable salt or buffering agent, such as, for example, sodium chloride, or sodium or potassium phosphate, respectively. Some examples of additives include, for example, glycerol, benzyl alcohol, and 1,1,1-trichloro-2-methyl-2-propanol (e.g., chloretone or chlorobutanol). If required, the pH of the solutions can be suitably adjusted by inclusion of a pH adjusting agent.

Compositions and formulations for parenteral, intrathecal, or intraventricular administration may include sterile aqueous solutions that may also contain buffers, diluents, and other suitable additives such as, but not limited to, penetration enhancers, carrier compounds, and other pharmaceutically acceptable carriers or excipients.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Pharmaceutical compositions of the present invention include, but are not limited to, solutions, emulsions, and liposome-containing formulations. These compositions may be generated from a variety of components that include, but are not limited to, preformed liquids, self-emulsifying solids, and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature, these formulations vary in the components and the consistency of the final product.

The pharmaceutical composition may or may not also include one or more additional pharmaceutically active or auxiliary compounds outside the scope of Formula (1). The additional active compound may, for example, suitably improve, augment, or otherwise suitably adjust the activity of the composition of Formula (1), or suitably adjust or diminish an undesired aspect of the composition of Formula (1), such as a side effect. In some embodiments, the one or more additional pharmaceutically active compounds may serve to treat any of the diseases described herein.

The pharmaceutical compositions of the present invention may additionally contain other adjunct or therapeutic components or agents conventionally found in pharmaceutical compositions. Thus, for example, the compositions may contain additional compatible pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, or salts for influencing osmotic pressure, buffers, colorings, flavorings and/or aromatic substances and the like, as long as they do not deleteriously interact with components of the formulation.

The invention further provides a kit comprising a composition according to Formula (1) in a pharmaceutically acceptable carrier. The kit can include any of the components typically used in the administration and use of a pharmaceutical. Thus, the kit may include any apparatus components necessary in the administration of the pharmaceutical, such as, for example, a packaged pharmaceutically acceptable dose of the pharmaceutical, instructions for use of the pharmaceutical, and accessories for administration, such as a needle or pad, if applicable, and optionally, any additional therapeutic agents to be co-administered to a subject.

Methods of Drug Delivery and Therapeutic Treatment

In another aspect, the invention is directed to targeted delivery of a drug to cells expressing higher levels of P-glycoprotein compared to other cells in a mammal. Such targeted drug delivery is useful for therapeutic treatment of diseases or conditions.

As the composition according to Formula (1) targets P-gp, cells with higher density of P-gp (e.g., cells in the liver, gastrointestinal epithelium, pancreas, kidney, colon, jejunum, epithelial blood-brain barrier and multi-drug resistant tumor) are the most effective targets. Thus, P-gp-targeted drug delivery compositions disclosed herein are particularly useful for delivery of drugs to or in the vicinity of cells and tissues with higher density of P-gp that are in need of treatment. In addition, in cases of targeted delivery of a drug to the GI tract (i.e., orally), the subsequent release of the drug from the carrier at the GI epithelium can treat either a local condition or disorder in or of the GI tract, or treats a condition remote from the GI tract where the drug is transported/pumped into the bloodstream and the targeted delivery of the drug to P-gp at the GI epithelium leads to a longer retention time in the GI epithelium to permit better transport into the bloodstream.

Once the p-gp-targeting composition described herein is docked at a P-gp site, the drug can be released by any of several mechanisms. For example, if the biocompatible framework carrying the drug is biodegradable, the framework will slowly or quickly degrade to release the drug. Moreover, the biocompatible framework may be tailored to include biodegradable groups particularly susceptible to enzymes located in biological tissue, such as disulfide or ester groups. In other embodiments, the biocompatible framework may be suitably porous to slowly release the drug over time. In yet other embodiments, the biocompatible framework may be tailored to be sufficiently water soluble to dissolve over time, or may be composed of a pH-activated material that releases the drug under biological pH conditions, as well known in the art, e.g., V. Balamuralidhara, et al., 2011, “pH Sensitive Drug Delivery Systems: A Review”, American Journal of Drug Discovery and Development, 1: 24-48.

The composition according to Formula (1) or pharmaceutical composition thereof of the present invention may be administered in a number of ways. Administration may be enteral (i.e., oral), topical (i.e., on the skin, including ophthalmic and to mucous membranes, including vaginal and rectal delivery), pulmonary (e.g., by inhalation or insufflation of powders or aerosols, including by nebulizer; intratracheal, intranasal, epidermal and transdermal), parenteral (i.e., by infusion through the skin), or by injection (e.g., intravenously or intramuscularly). Parenteral administration includes intravenous, intraarterial, subcutaneous, intraperitoneal or intramuscular injection or infusion; or intracranial, e.g., intrathecal or intraventricular, administration. For oral administration, liquid or solid oral formulations can be given. These include, for example, tablets, capsules, pills, troches, elixirs, suspensions, and syrups.

Dosing is dependent on the severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates.

The composition according to Formula (1) is administered in a pharmaceutically effective (i.e., treatment-effective) amount, which is an amount suitable for effecting an observable favorable change in the course of the disease or condition. Optimum dosages may vary depending on the relative potency of individual compounds, and can generally be estimated based on EC₅₀ or IC₅₀ values found to be effective in in vitro and in vivo animal models or based on the examples described herein. In general, dosage is from 0.01 μg to 100 g per kg of body weight, and may be given once or more daily, weekly, monthly, or yearly. In different embodiments, the composition according to Formula (1) is administered at a dosage of precisely, about, at least, above, up to, or less than, for example, 5 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 600 mg, 700 mg, 800 mg, 900 mg, 1000 mg, 1200 mg, or 1500 mg per administration, wherein the compound can be administered by any suitable schedule, e.g., once daily, once weekly, twice daily, or twice weekly. The composition according to Formula (1) can also be administered in a way which releases the compound into the subject in a controlled manner over time (i.e., as a controlled release formulation), by means well known in the art, such as by use of a time release capsule or time-releasing (e.g., slow dissolving) physical form of the compound. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the therapy is administered in maintenance doses, ranging from 0.01 μg to 100 g per kg of body weight, once or more daily, for a suitable time period.

The composition according to Formula (1) can be co-administered with one or more other therapeutic agents outside the scope of Formula (1). In particular, an antineoplastic (e.g., anticancer) agent can be included in the treatment, either as a drug in the composition according to Formula (1) and/or separate and apart from the composition according to Formula (1). Anticancer agents suitable for use with the present invention include, but are not limited to, agents that induce apoptosis, agents that inhibit adenosine deaminase function, inhibit pyrimidine biosynthesis, inhibit purine ring biosynthesis, inhibit nucleotide interconversions, inhibit ribonucleotide reductase, inhibit thymidine monophosphate (TMP) synthesis, inhibit dihydrofolate reduction, inhibit DNA synthesis, form adducts with DNA, damage DNA, inhibit DNA repair, intercalate with DNA, deaminate asparagines, inhibit RNA synthesis, inhibit protein synthesis or stability, inhibit microtubule synthesis or function, inhibit protein kinase activity, block receptors for growth factors, cytokines, activating ligands, and the like.

In one embodiment, the disease or condition being treated is cancer. The cancer can be, for example, colon cancer, gastrointestinal cancer, lung cancer, pancreatic cancer, bladder cancer, breast cancer, ovarian cancer, esophageal cancer, head-and-neck cancer, skin cancer, brain cancer, diffuse large cell lymphoma, follicular B cell lymphoma, lymphocytic leukemia, multiple myeloma, Burkitt's lymphoma, primary mediastinal B-cell lymphoma, Hodgkin's lymphoma, non-Hodgkin's lymphoma, mantle cell lymphoma, Kaposi's sarcoma, and Cowden's syndrome. In particular embodiments, the cancer is characterized by a multidrug resistant (MDR) tumor.

In another embodiment, the disease or condition being treated is a neurological disease or condition. The neurological disease or condition can be, for example, neurodegenerative disorders, such as Alzheimer's disease, Parkinson's disease, and Huntington's disease; Tourette's syndrome, depression, anxiety, psychosis, paranoia, schizophrenia, sleep disorder, eating disorder, compulsive disorders, addictive disorders and pain.

In another embodiment, the disease or condition being treated is a gastrointestinal or digestive disease or condition. The gastrointestinal disease or condition can be, for example, gastroesophageal reflux disease (GERD), esophagitis, esophageal motility disorder, gastritis, peptic ulcer, dyspepsia, enterocolitis, Crohn's disease, diarrhea, enteritis, colitis, diverticulitis, functional colonic disease, and celiac disease.

In another embodiment, the disease or condition being treated is an infection, wherein the infection is caused by a microbe, such as a bacteria, virus, fungus, or parasite. The bacterial infection can be a result of, for example, a bacterium of the Bacilli, Cocci, Spirochaetes, or Vibrio class. The viral infection can be a result of, for example, a rhino virus, influenza, chicken pox, herpes, or retrovirus (e.g., HIV).

Other diseases and conditions suitable for treatment herein include, e.g., inflammation, an elevated level of cholesterol (e.g., hypercholesterolemia), diabetes, hypertension, a cardiovascular disease or condition, obesity, and muscular dystrophy.

In a particular embodiment, the composition according to Formula (1) includes a psychoactive drug that would normally (i.e., in unconjugated form) be prevented or substantially hindered from penetrating the blood-brain membrane by virtue of the high density of P-gp in epithelial cells guarding the blood-brain barrier, i.e., A. H. Schinkel, “P-Glycoprotein, a gatekeeper in the blood-brain barrier”, Adv. Drug Deliv. Rev., 36(2-3), 1999, pp. 179-194, the contents of which are herein incorporated by reference in their entirety. By virtue of the P-gp substrate in the composition of Formula (1), the resulting composition will target the blood-brain barrier, thereby releasing the psychoactive drug at the vicinity of the blood-brain barrier, which can result in significantly improved penetration across the blood-brain barrier. Thus, psychoactive drugs that may have heretofore been in disuse for their substantial inability to cross the blood-brain barrier could now benefit by their incorporation into the composition of Formula (1) with subsequent administration into a subject. By analogous means, the drug to be passed through the blood-brain barrier may be a drug useful in treating a non-psychoactive disease or condition of the brain, such as brain cancer or a cerebrovascular blockage (stroke).

The present description is further illustrated by the following examples, which should not be construed as limiting in any way. The contents of all cited references (including literature references, issued patents, and published patent applications as cited throughout this application) are hereby expressly incorporated by reference.

### Example-1

### Design and Creation of Biotin-PEG-Rho123

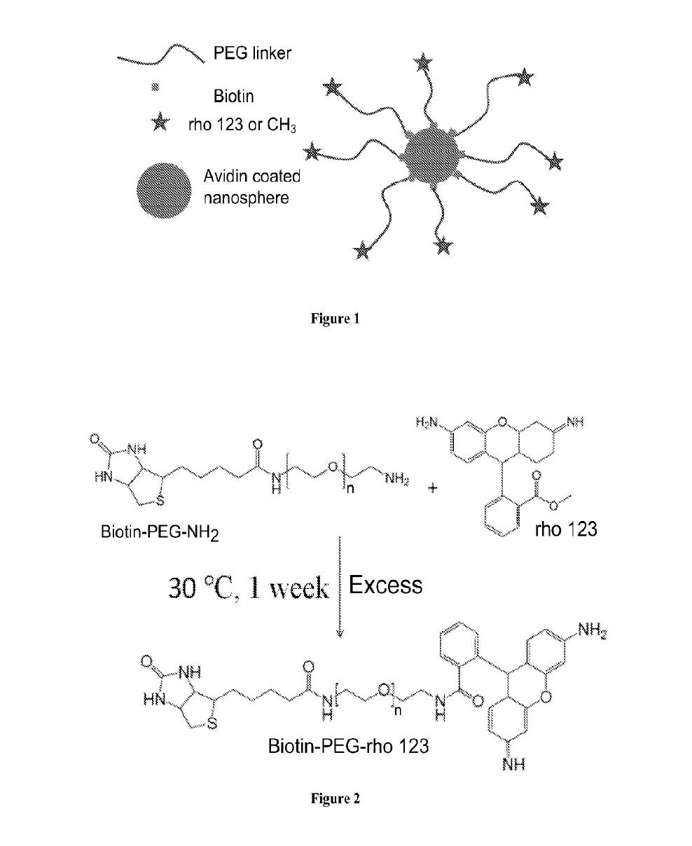

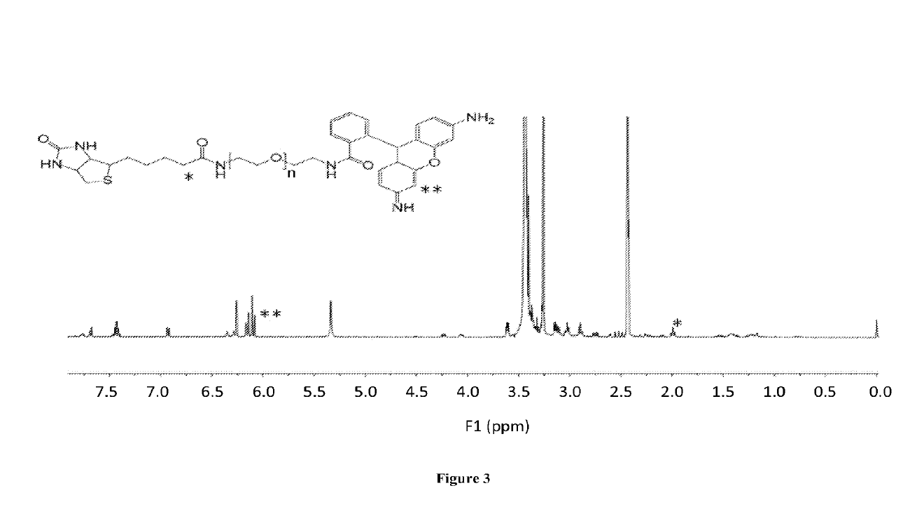

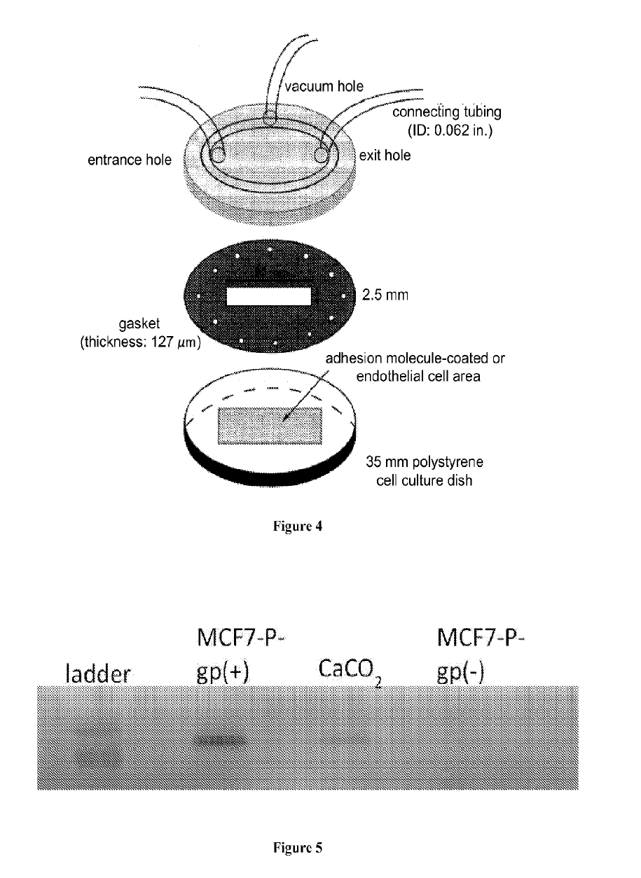

The delivery system of this Example consisted of the biotin-PEG-Rho123 conjugate attached via the avidin-biotin link to fluorescently labeled avidin-coated polystyrene beads. The beads allow the visualization of the system by fluorescent microscopy thereby allowing the quantitation of binding in the follow-on flow chamber experiments as described below. A cartoon representation of the final conjugate is provided in FIG. 1. The synthesis of PEG (Mw 7500) tethered to the P-gp substrate, rhodamine123 was conducted as shown in FIG. 2. Confirmation of the conjugate structure was determined by 1H NMR (FIG. 3).

Flow chamber experiments were conducted to determine the binding capacity of the P-gp particles to P-gp expressing breast cancer cells compared to non-P-gp expressing breast cancer cells. The configuration of the flow chamber is provided in FIG. 4.

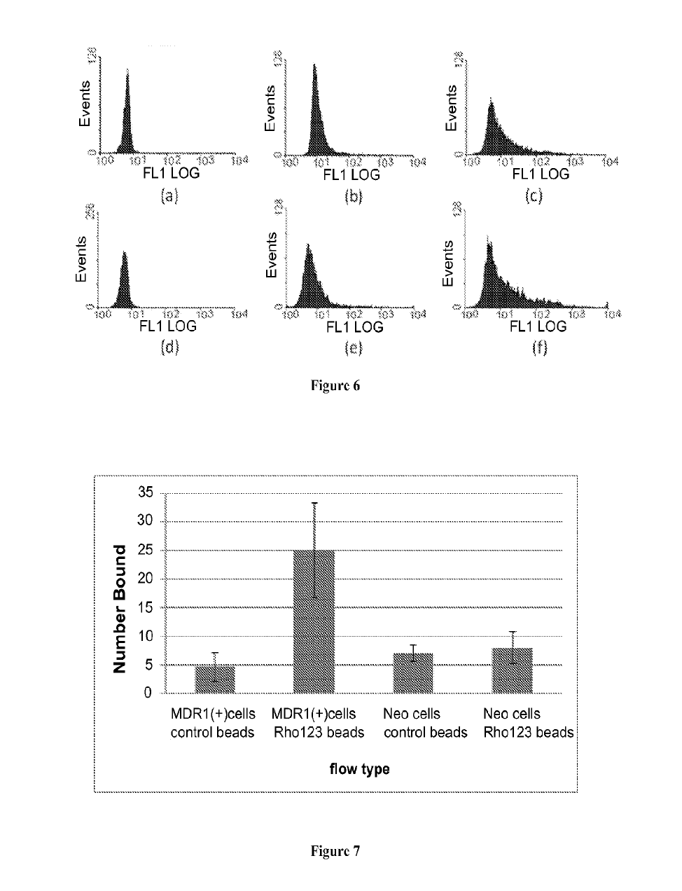

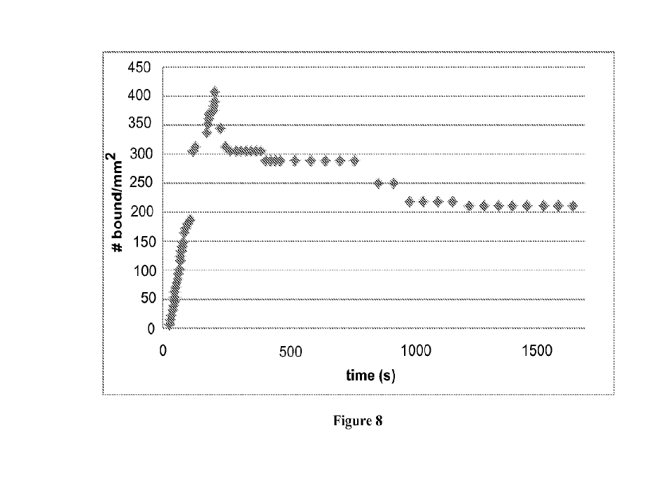

The MCF-7 breast adenocarcinoma cell line, engineered to either express P-gp (MCF-7/Mdr1) or not express P-gp (MCF-7/wt) were grown on the surface of a glass plate. Expression or absence of P-gp in each cell line was determined by Western blot analysis (FIG. 5). FACS analysis was used to quantify the number of targeted and untargeted beads to both P-gp expressing and P-gp non-expression cells (FIG. 6). From the flow chamber data, the total number of bound beads was quantified (experiments conducted in triplicate). The output from the experiment is shown in FIG. 7.

Lastly, the kinetics of rhodamine binding to MDR(+) cells (i.e., MCF-7/Mdr1) was determined over a 30 min timeframe (FIG. 8). The data reveal a rapid binding curve followed by an approximately first-order elimination followed by a steady state concentration. These data suggest a potential validation of the “fishing hook” hypothesis for how these conjugates bind to P-gp(+) cell lines.

The conjugation of a P-gp substrate to PEG-biotin allowed the formulation of polystyrene beads with the ability to selectively bind to P-gp(+) cells. The degree of binding was successfully quantified using a flow chamber mounted to a fluorescent microscope and FACS. Collectively, these data suggest that a P-gp substrate can be successfully used to selectively target P-gp(+) cell types.

### Example-2

### Design, Synthesis and Characterization of mPEG-Rho123

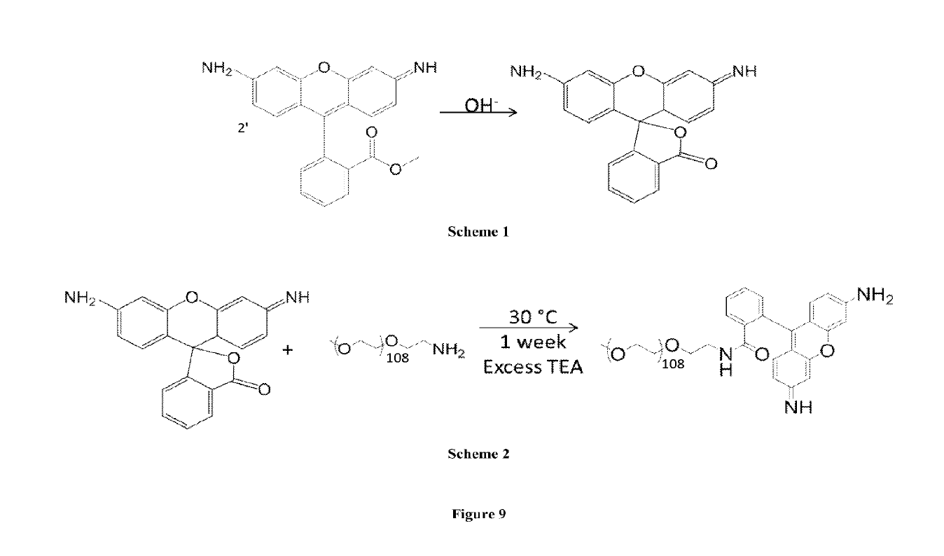

The synthesis of the mPEG-Rho 123 conjugate was achieved according to Scheme 2 (FIG. 9) and adapted from the report by ADAMCZYK, et al. (J. Bioorganic & Medicinal Chemistry Letters, 10: 1539-41 (2000)) describing a one-step substitution of small compounds at the 2′ position of Rho123 and Rho6G (see Scheme 1 in FIG. 9).

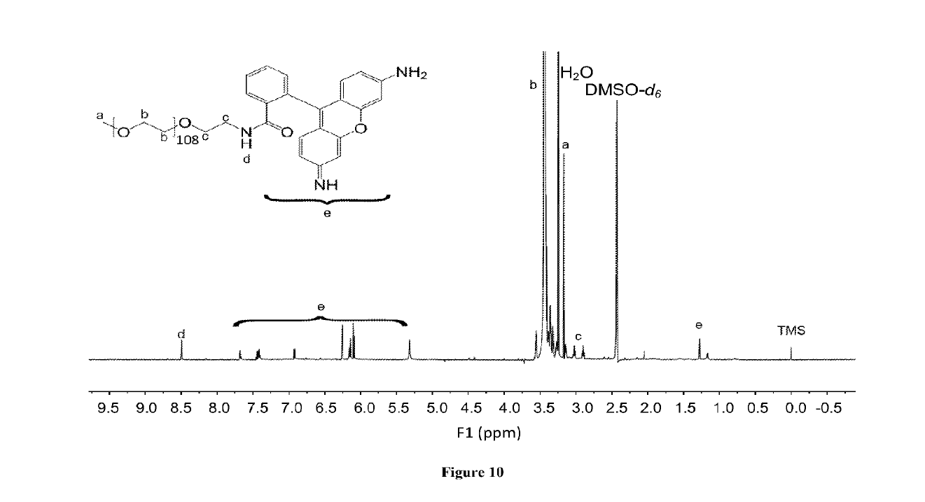

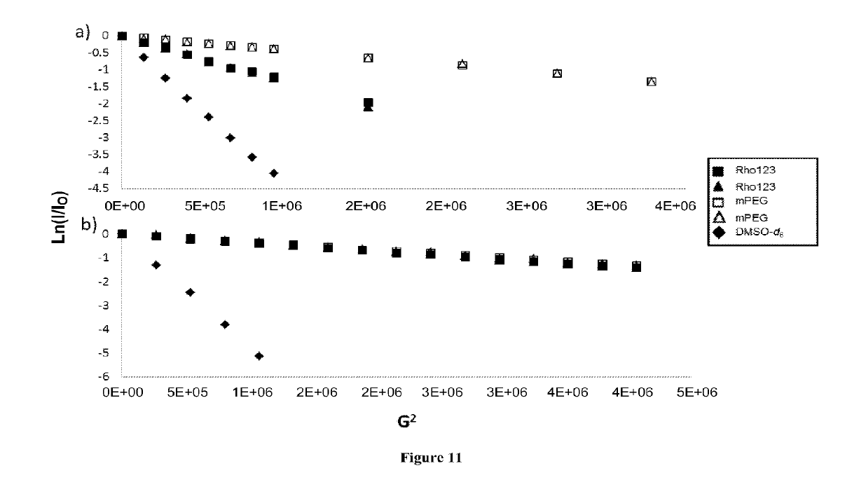

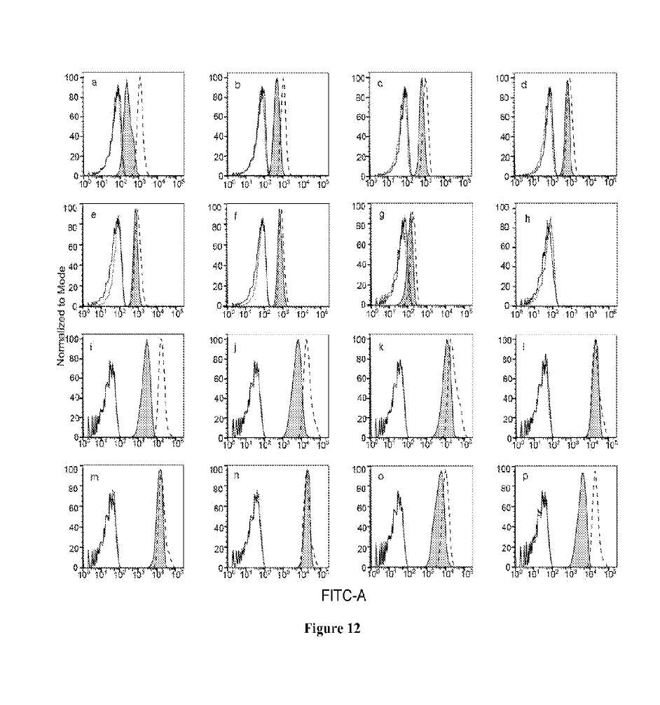

The conjugate of mPEG-Rho123 was characterized by ¹H NMR and diffusion ordered NMR (FIG. 10 and FIG. 11, respectively). The single-proton NMR of the mPEG-Rho123 conjugate (FIG. 10) shows the Rho123 peaks visible from a chemical shift between 5 and 8 ppm (aromatic region). Percent conjugation yield was 65% and was calculated from peaks at 3.17 (from mPEG), 6.14 and 6.16 (from Rho123) ppm. To confirm that the ¹H NMR results were not from an unconjugated mixture of mPEG-NH₂ and free Rho123 and that a covalent conjugate was made, diffusion ordered NMR (which calculates relative diffusion of each peak in the spectra) was also performed (FIG. 11).

Small molecules, like free Rho123, diffuse fast relative to macromolecules, like mPEG-NH₂. NMR signals with equal diffusion rates are indicative of covalently linked compounds. Diffusion ordered spectras of free Rho 123 and unconjugated mPEG-NH₂ (FIG. 11a) and of conjugated mPEG-Rho123 (FIG. 11b) were obtained and represented by Stejskals-Tanner plots. Both the mPEG and Rho123 peaks were present and identifiable on the ¹H spectra and the diffusion ordered NMR confirmed covalent conjugation. When free Rho123 is physically mixed with unconjugated mPEG-NH₂, the decrease in peak intensity in the diffusion ordered NMR is much faster for Rho 123 peaks than for mPEG-NH₂ peaks, which translates into very different diffusion rates and depicts a lack of covalent attachment. However, once Rho123 is conjugated to the mPEG, the decrease in intensity is the same for both the Rho123 and mPEG peaks, which depicts covalent attachment of the compounds. The behavior of faster decay for smaller molecules seen on the Stejskals-Tanner plots is a consistent result given the size of the molecules involved; the smaller the molecule, the faster the signal decays with increasing gradient (JOHNSON, Jr., Prog. Nucl. Magn. Reson. Spectrosc. 1999, 34: 203-256 (1999); MOMOT, et al., Concepts in Magnetic Resonance, 19A: 51-64 (2003); KAUCHER, et al., Chemistry, 11: 164-73 (2004)).

The results show definitively that Rho123 was covalently conjugated to the mPEG-NH₂ tether.

Once conjugation of mPEG-Rho 123 was achieved, the conjugate was evaluated for P-gp interactions. The interaction between the mPEG-Rho123 conjugate and P-gp was investigated using two MDA-435/LCC6 cell lines: MDA-435/LCC6 MDR which expresses P-gp, and MDA-435/LCC6 WT which does not (LEONESSAL, et al., British Journal of Cancer, 161: 154-161 (1996)). Before tests were initiated, P-gp expression in both MDR and WT cells was confirmed by Western blot, which confirmed the expression of P-gp in the MDR cells and the lack of expression in WT cells. The protein band in the MDR cells was visible around the molecular weight of P-gp, 170 kDa, which correlates to the reported molecular weight of the protein.

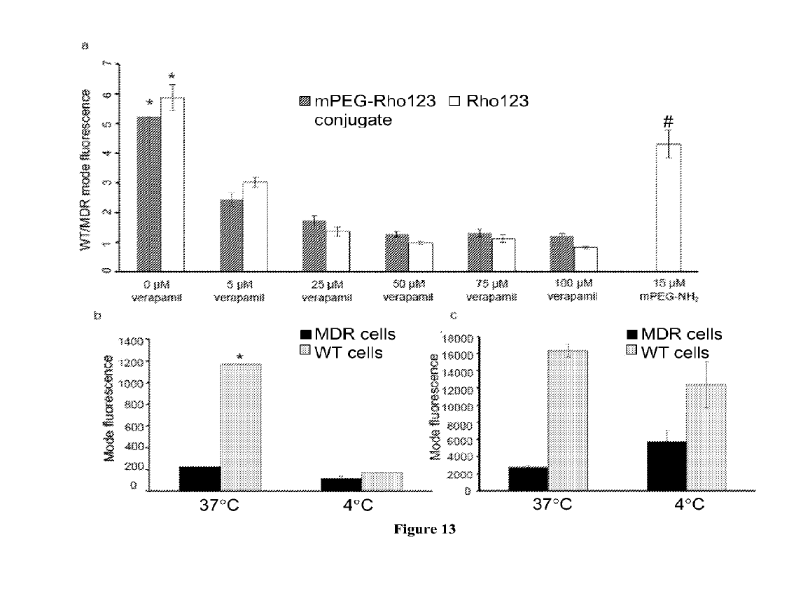

Representative quantification of mPEG-Rho 123 accumulation in MDR and WT cells was obtained via FACS (Fluorescent Assisted Cell Sorting) experiments (FIGS. 12-13). Initial FACS experiments were conducted at 37° C. with 0 μM verapamil to investigate the efflux of both mPEG-Rho123 conjugate and free Rho123 from MDR cells compared with WT cells (FIGS. 12a and 12i, respectively). To show that the mechanism of efflux was mediated by P-gp a series of verapamil (a well-established P-gp inhibitor (TSURUO, et al., Biochem. Pharmacol., 31: 3138-40 (1982)) concentrations (5, 25, 50, 75 and 100 μM) were added to the incubations (FIGS. 12b-f and 12j-n). Additionally, since P-gp is an ATP driven protein and recognizing that the internalization pathway of the mPEG-Rho123 conjugate is likely by endocytosis, FACS experiments were also conducted at 4° C. to inhibit both P-gp and endocytosis activity (FIGS. 12g and 12o). To ensure that unconjugated mPEG-NH₂ had no influence on the results, the free polymer was incubated alone and with free Rho123 in both MDR and WT cells (FIGS. 12h and 12p). No differences in P-gp function were observed. FIG. 13 shows the quantitated ratio between fluorescence signals from MDR and WT cells, verifying the trends visually represented in FIG. 12. Statistics represent the significance differences in WT/MDR accumulation ratio at a p-value of <0.01.

Visual verification of the FACS results was obtained by confocal microscopy of cells incubated with both mPEG-Rho123 conjugate and free Rho123. Images were taken of mPEG-Rho123 conjugate and free Rho123 incubated with MDR and WT cells at 37° C. with 0 μM of verapamil P-gp inhibitor. Images were also obtained from identical incubations conducted in the presence of 25 μM of verapamil to observe the increase in accumulation of mPEG-Rho123 conjugate and free Rho123.

Initial FACS experiments at 37° C. and 0 μM verapamil showed similar efflux patterns for both free Rho123 and the mPEG-Rho123 conjugate. These results correlate well with previous reports that show how free Rho123 interacted with MDR expressing cells relative to WT cells (LUDESCHER, et al., J. British Journal of Haematology, 82: 161-168 (1992); ROBEY, et al., Blood, 93: 306-314 (1999)). The mPEG-Rho123 conjugate follows the same efflux pattern as free Rho123, showing that the mPEG-Rho123 conjugate remains a substrate of P-gp. This Example also shows that conjugation of mPEG at the 2′ methyl ester site of Rho123 decreased its quantum yield, which caused the fluorescent readings observed in FACS to be shifted lower than free Rho123; a result seen previously for Rhodamine 6G (ADAMCZYK, et al., Bioorganic & Medicinal Chemistry Letters, 13: 2327-2330 (2003)). The ratio of WT to MDR accumulation shows that there is no statistical difference between efflux at 37° C. with 0 μM verapamil for the mPEG-Rho123 conjugate and free Rho123. Confirmation of P-gp mediated efflux of the mPEG-Rho 123 conjugate was accomplished by serial increases in the concentration of verapamil. Verapamil is a well-established inhibitor of P-gp that increases intracellular accumulation of free Rho 123 in a concentration dependent manner. Higher concentrations of verapamil lead to higher accumulation of Rho123 in MDR expressing cells (AGUERO, et al., Journal of Clinical Laser Medicine & Surgery, 12: 193-198 (1994); YUMOTO, et al., The Journal of Pharmacology and Experimental Therapeutics, 289: 149-55 (1999)). This trend was also observed in this study for both free Rho123 and the mPEG-Rho123 conjugate. In both cases, there was little change in signal after 50 μM of verapamil which is attributed to the toxicity of the verapamil at higher concentrations.

The effects of the mPEG-NH₂ on P-gp efflux efficacy were also determined to account for potential effects of residual unconjugated polymer in the preparation. P-gp inhibitory effects of PEG and PEG-containing copolymers have been previously reported (SHEN, et al., Int. J. Pharm., 313: 49-56 (2006); HUGGER, et al., J. Pharm. Sci., 91: 1991-2002 (2002); JOHNSON, et al., AAPS PharmSci, 4: E40 (2002)). However, the reported concentration ranges at which inhibition occurs are on the order of 10⁻⁴ to 10⁻² M, which is one or more orders of magnitude greater than the calculated 15 μM of unconjugated mPEG-NH₂ in these experiments (WERLE, Pharm. Res., 25: 500-511 (2008)). The physical mixture of Rho123 with mPEG-NH₂ showed statistically insignificant inhibitory effects on Rho123 efflux, signifying that the unconjugated polymer did not account for the mPEG-Rho 123 results.

Since P-gp mediated efflux is an ATP driven process, the incubation of MDA-435/LCC6 MDR cells at 4° C. inhibits its efflux activity, as well as endocytosis, and the transport of substrates is significantly reduced (SHAROM, et al., Biochem. J., 308: 381-390 (1995)). Results from efflux studies conducted at 4° C. show an increase in free Rho123 accumulation in MDR cells relative to the same experiment conducted at 37° C. In the same experiment with the mPEG-Rho123 conjugate the MDR and WT spectras closely overlapped and the accumulation in MDR cells approached accumulation in WT cells, a result indicative of P-gp efflux activity inhibition. However, accumulation in WT cells is significantly decreased from accumulation at 37° C. signifying endocytosis is the mechanism of cell internalization for the mPEG-Rho123 conjugate. Endocytosis is a pathway that has been described as a mode of entry for macromolecules into intracellular space (WERLE, Pharm. Res., 25: 500-11 (2008); KOPECEK, et al., Controlled Release, 74: 147-158 (2001); KOPECEK, et al., European Journal of Pharmaceutics and Biopharmaceutics, 50: 61-81 (2000); CHRISTIAN DE DUVE, et al., Biochem. Pharmacol., 23: 2495-2531 (1974)) and P-gp has also been shown to undergo endocytosis and retain activity (F U, et al., The International Journal of Biochemistry and Cell Biology, 44: 461-464 (2012)).

### Example-3

### Synthesis of PLA-mPEG Diblock Copolymer and Nanoparticle Formulation

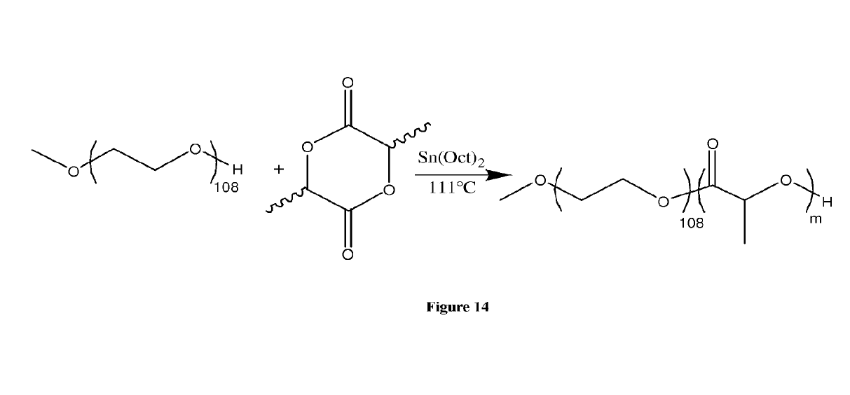

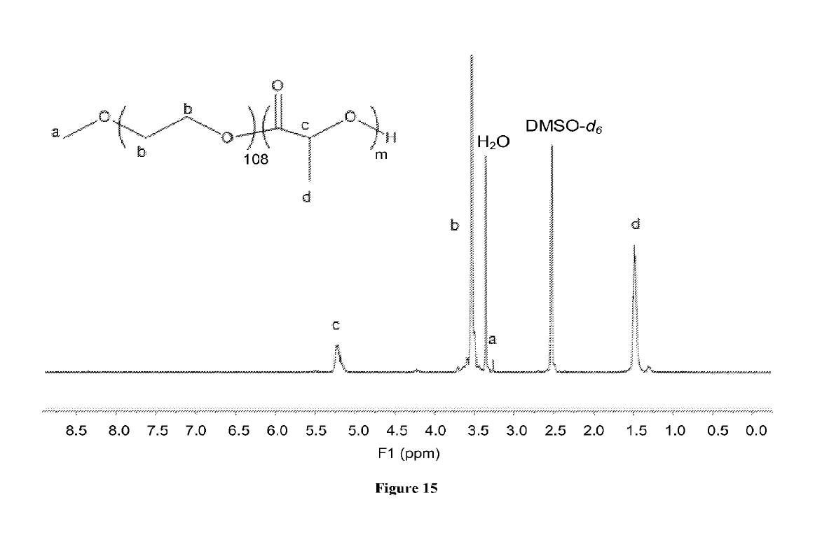

PLA-mPEG diblock copolymer was synthesized by ring opening polymerization of D,L-lactide by an already polymerized mPEG block (5000 MO (Scheme 3, FIG. 14). The various mPEG-PLA polymers synthesized are listed in Table 1. A range of polylactide Mₙ values were synthesized to evaluate the polymer that would give a nanoparticle around 100 nm. Molecular weights were determined with ¹H NMR (FIG. 15) using end group analysis and GPC was performed to confirm that the polymerization of lactide was due to the mPEG chain and to determine the polydispersity index (PDI) of the polymer.

TABLE 1
Summary of PLA-mPEG polymers made and nanoparticles produced from each polymer.

| | Mol mPEG | Mol | Mₙ PLA from | PDI from | Particle size | Particle |
|---|---|---|---|---|---|---|
| Entry | (5000 Mₙ) | Lactide | NMR | GPC | (nm) | PDI |
| 1 | 1.1 × 10⁻⁴ | 2.4 × 10⁻³ | 2,640 | 1.13 | 28.0 ± .3 | 0.116 ± 0.02 |
| 2 | 5 × 10⁻⁵ | 2.6 × 10⁻³ | 4,708 | 1.12 | 37.7 ± 2.9 | 0.162 ± 0.03 |
| 3 | 2 × 10⁻⁵ | 3.2 × 10⁻³ | 12,364 | 1.3 | 52.1 ± 3 | 0.22 ± 0.01 |
| 4 | 1 × 10⁻⁵ | 7.72 × 10⁻³ | 73,129 | 1.6 | 88.1 ± 13 | 0.24 ± 0.07 |

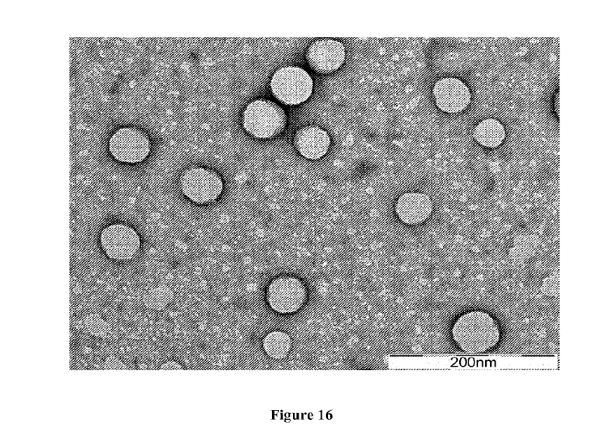

Nanoparticles were synthesized by the nanoprecipitation technique (FESSI, et al., Int. J. Pharm., 55: 1-4 (1989)). The polymer was first dissolved in acetone then added drop-wise to stirring water. The acetone was then evaporated and the particles were purified by centrifugation. The particle size was determined by dynamic light scattering (Table 2). Images were taken of the largest particles using TEM to determine particle morphology (FIG. 16).

PLA-mPEG was synthesized at various PLA Mₙ values and each polymer was used to produce nanoparticles of various sizes. The pattern of increasing PLA size with increasing polymer size has been shown in literature (WANG, et al., Pharm. Res., 27: 1861-1868 (2010); RILEY, et al., Colloids Surf B, 16: 147-159 (1999)). A particle of around 100 nm in diameter has been produced from mPEG:PLA copolymers of 5000:75,000 molecular weights (GOVENDER, et al., Int. J. Pharm., 199: 95-110 (2000); RILEY, et al., Langmuir, 17: 3168-3174 (2001)). The 5000:73,000 polymer produced particles close to 100 nm. The TEM image shows that the particles were spherical in morphology, consistent with previous reports using the nanoprecipitation technique (FESSI, et al., Int. J. Pharm., 55: 1-4 (1989); CHORNY, et al., Controlled Release, 83: 389-400 (2002)).

### Example-4

### Initial Biodistribution Studies

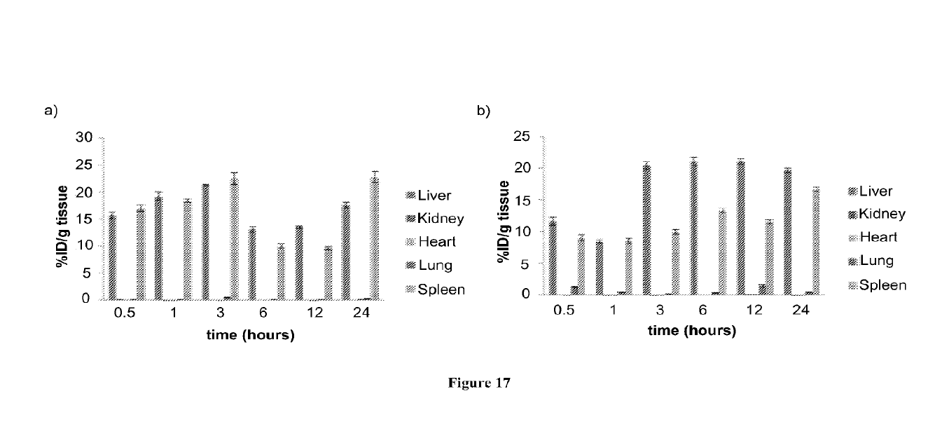

Initial biodistribution studies were done with model polystyrene particles. Europium polystyrene nanoparticles were commercially available and used to test biodistribution detection. The particles purchased were coated in avidin which allowed testing of an mPEG corona and PEG-Rho123 corona through the biotin-avidin interaction. Biotin-mPEG was purchased and biotin-PEG-Rho 123 was synthesized using the same method as mPEG-Rho123. Europium nanoparticles were injected via tail vein injection into nude mice. At appropriate times the mice were euthanized and the liver, kidney, lungs, spleen and heart were collected. Each organ was weighed and homogenized in 2 mL of RIPA cell lysis buffer. Time resolved fluorescence was measured on a fluorescent plate reader and compared to the fluorescence of injected dose. Results are presented in FIG. 17.

These results from initial biodistribution studies show that europium is a suitable choice for tracking particles in vivo. Particle levels were detectable and differences in surface coating could be detected.

### Example-5

### Initial Xenograft Development

Xenografts with both MDA-435/LCC6 MDR and MDA-435/LCC6 WT cells were developed in 6 week old immunodeficient Nu/Nu mice. 1-2 million cells were injected subcutaneously into the back haunches of 36 mice for each cell type. Tumor measurements were taken with vernier calipers. The volume was calculated using a modified ellipsoidal equation (EUHUS, et al. Journal of Surgical Oncology, 31: 229-34 (1986); TOMAYKO, et al., Cancer Chemotherapy and Pharmacology, 24: 148-154 (1989); JENSEN, et al., BMC Medical Imaging, 8: 16 (2008)) (Equation 1).

V=0.5*l*w² (Eq. 1)

Growth curves show successful growth of tumors for both MDR and WT cells starting at 7 days after subcutaneous injection of cells. PLA-PEG particles described above are tested for biodistribution in these mice.

What is claimed is:

1. A composition having the following general structure: 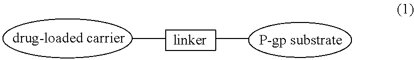
wherein:
said P-gp substrate is a substrate for P-glycoprotein;
said linker is a biocompatible polymeric moiety;
said drug-loaded carrier comprises a biocompatible framework carrying at least one drug;
and the straight line shown in Formula (1) between the drug-loaded carrier and linker represents a first bond, and the straight line shown in Formula (1) between the linker and P-gp substrate represents a second bond.

2. The composition of claim 1, wherein said drug is encapsulated in, intercalated in, embedded in, absorbed to, or conjugated to said biocompatible framework in said drug-loaded carrier.

3. The composition of claim 1, wherein said drug is attached to an outer surface of said biocompatible framework in said drug-loaded carrier.

4. The composition of claim 1, wherein said biocompatible framework comprises a biocompatible polymer, liposome, or micelle.

5. The composition of claim 4, wherein said biocompatible polymer is selected from polyhydroxyacid biopolyesters, polysaccharides, vinyl addition polymers, polyalkyleneglycols, polyphosphazenes, polyanhydrides, polyacetals, poly(ortho esters), polyureas, polyurethanes, polyamides, poly(amino acids), polyphosphoesters, and co-polymers thereof.

6. The composition of claim 4, wherein said biocompatible polymer comprises a polyhydroxyacid biopolyester.

7. The composition of claim 6, wherein said polyhydroxyacid biopolyester is selected from poly(α-hydroxy acid)s and poly(hydroxyalkanoates).

8. The composition of claim 7, wherein said poly(α-hydroxy acid)s are selected from polylactic acid, polyglycolic acid, and copolymers thereof.

9. The composition of claim 7, wherein said poly(hydroxyalkanoates) are selected from poly(3-hydroxypropionate), poly(3-hydroxybutyrate), poly(4-hydroxybutyrate), poly(3-hydroxyvalerate), poly(4-hydroxyvalerate), poly(5-hydroxyvalerate), poly(ε-caprolactone), poly(3-hydroxyhexanoate), poly(3-hydroxyoctanoate), and copolymers thereof.

10. The composition of claim 1, wherein said linker has a length sufficient for at least partially traversing a cell membrane.

11. The composition of claim 10, wherein said length sufficient for at least partially traversing a cell membrane is at least 3 nm.

12. The composition of claim 1, wherein said linker comprises a polymer block of at least 6 ethyleneoxy units.

13. The composition of claim 1, wherein said substrate selectively targets cells having a higher density of P-glycoprotein compared to other cells in a mammal.

14. The composition of claim 1, wherein said drug comprises an anti-cancer drug.

15. The composition of claim 1, wherein said drug comprises a neuroactive drug.

16. The composition of claim 1, wherein said drug comprises a gastrointestinal agent.

17. The composition of claim 1, wherein said drug comprises an antibiotic or antiviral agent.

18. A pharmaceutical composition comprising the composition of claim 1 in a pharmaceutically acceptable carrier.

19. A method for targeted delivery of a drug to cells expressing higher levels of P-glycoprotein compared to other cells in a mammal, comprising administering to said mammal a pharmaceutically effective amount of a composition having the following general structure: 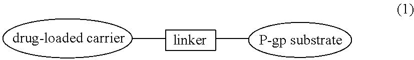
wherein:
said P-gp substrate is a substrate for P-glycoprotein;
said linker is a biocompatible polymeric moiety;
said drug-loaded carrier comprises a biocompatible framework carrying at least one drug;
and the straight line shown in Formula (1) between the drug-loaded carrier and linker represents a first bond, and the straight line shown in Formula (1) between the linker and P-gp substrate represents a second bond.

20. The method of claim 19, wherein said cells are cancerous cells, and said drug is an anti-cancer drug.

21. The method of claim 20, wherein said cancerous cells are multi-drug resistant cancerous cells.

22. The method of claim 19, wherein said cells are cells of the central nervous system of said mammal.

23. The method of claim 22, wherein said mammal suffers from a neurological disease, and said drug is a neuroactive drug.

24. The method of claim 23, wherein said neuroactive drug has an ability to cross a blood-brain barrier.

25. The method of claim 23, wherein said neurological disease is selected from the group consisting of Parkinson's disease, Alzheimer's disease, Huntington's disease, pain and depression.

26. The method of claim 19, wherein said cells are cells of the gastrointestinal epithelium.

27. The method of claim 19, wherein said mammal suffers from a disease or condition affecting the gastrointestinal tract.

28. The method of claim 19, wherein said disease or condition is treated systemically with a drug administered orally.
